(19) United States
(12) Patent Application Publication
(10) Pub. No.: US 2011/0092558 A1
(43) Pub. Date: Apr. 21, 2011

(54) NOVEL MICROBIOCIDES

(75) Inventors: Daniel Stierli, Stein (CH); Harald Walter, Stein (CH); Ramya Rajan, Goa (IN)

(73) Assignee: SYNGENTA CROP PROTECTION, INC., Greensboro, NC (US)

(21) Appl. No.: 12/988,460

(22) PCT Filed: Apr. 17, 2009
(86) PCT No.: PCT/EP2009/054586

§ 371 Date: Jan. 5, 2011

(30) Foreign Application Priority Data
Apr. 18, 2008 (GB) 0807140.9

(51) Int. Cl. C07D 231/14; A01N 43/56; C07D 419/02; A01P 3/00

(52) U.S. Cl. 514/406; 548/374.1; 544/2

(57) ABSTRACT

Compounds of formula (I) in which the substituents are as defined in claim 1, are suitable for use as microbiocides.

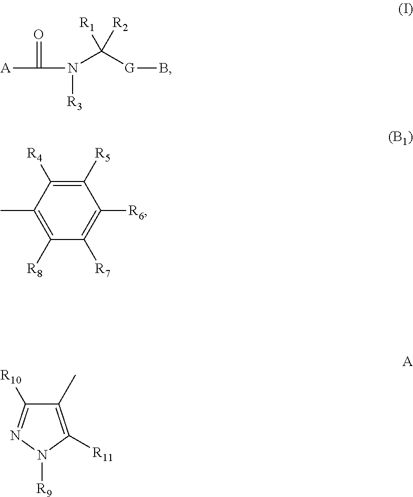

The present invention relates to novel microbiocidally active, in particular fungicidally active, carboxamides. It further relates to intermediates used in the preparation of these compounds, to compositions which comprise these compounds and to their use in agriculture or horticulture for controlling or preventing infestation of plants by phytopathogenic microorganisms, preferably fungi.

Fungicidally active N-(4-pyridin-2-ylbutyl)benzamide derivatives are described for example in WO 2008/003745 and WO 2007/141009. It has been found that novel carboxamides have microbiocidal activity.

The present invention accordingly relates to compounds of formula I

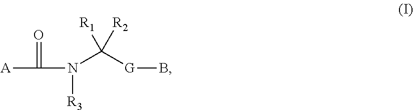

wherein
R₁ is hydrogen or C₁-C₄alkyl; R₂ is hydrogen or C₁-C₄alkyl;
R₃ is hydrogen or C₃-C₇cycloalkyl;
B is the group B₁

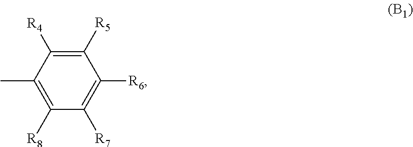

wherein
R₄ is hydrogen, halogen or C₁-C₆alkyl;
R₅ is hydrogen or halogen;
R₆ is hydrogen, halogen, C₁-C₆alkyl, C₂-C₆alkenyl, C₃-C₆alkinyl, C₃-C₆cycloalkyl-C₃-C₆alkinyl, halophenoxy, halophenyl-C₃-C₆alkinyl, C(C₁-C₄alkyl)═NO—C₁-C₄alkyl, C₁-C₆haloalkyl, C₁-C₆haloalkoxy, C₂-C₆haloalkenyl, or C₂-C₆haloalkenyloxy;
R₇ is hydrogen or halogen;
R₈ is hydrogen, halogen, C₁-C₆alkyl, C₁-C₆alkoxy,
G is selected from the group consisting of (CH₂)₂, (CH₂)₃, (CH₂)₂O and CH₂OCH₂;
A is the group A₁

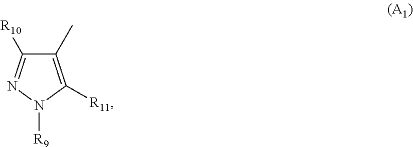

wherein
R₉ is hydrogen or C₁-C₄alkyl;
R₁₀ is C₁-C₄alkyl, C₁-C₄haloalkyl or halogen;
R₁₁ is hydrogen or halogen; and agronomically acceptable salts/isomers/structural isomers/stereoisomers/diastereoisomers/enantio-mers/tautomers and N-oxides of those compounds.

The alkyl groups occurring in the definitions of the substituents can be straight-chain or branched and are, for example, methyl, ethyl, n-propyl, n-butyl, n-pentyl, n-hexyl, iso-propyl, n-butyl, sec-butyl, iso-butyl or tert-butyl. Alkoxy, alkenyl and alkynyl radicals are derived from the alkyl radicals mentioned. The alkenyl and alkynyl groups can be mono- or di-unsaturated. The cycloalkyl groups occurring in the definitions of the substituents are, for example, cyclopropyl, cyclobutyl, cyclopentyl, cyclohexyl or cycloheptyl. Halogen is generally fluorine, chlorine, bromine or iodine, preferably fluorine, bromine or chlorine. This also applies, correspondingly, to halogen in combination with other meanings, such as halogenalkyl or halogenalkoxy. Haloalkyl groups preferably have a chain length of from 1 to 4 carbon atoms. Halonalkyl is, for example, fluoromethyl, difluoromethyl, trifluoromethyl, chloromethyl, dichloromethyl, trichloromethyl, 2,2,2-trifluoroethyl, 2-fluoroethyl, 2-chloroethyl, pentafluoroethyl, 1,1-difluoro-2,2,2-trichloroethyl, 2,2,3,3-tetrafluoroethyl and 2,2,2-trichloroethyl; preferably trichloromethyl, difluorochloromethyl, difluoromethyl, trifluoromethyl and dichlorofluoromethyl. Alkoxy is, for example, methoxy, ethoxy, propoxy, i-propoxy, n-butoxy, isobutoxy, sec-butoxy and tert-butoxy; preferably methoxy and ethoxy. Halogenalkoxy is, for example, fluoromethoxy, difluoromethoxy, trifluoromethoxy, 2,2,2-trifluoroethoxy, 1,1,2,2-tetrafluoroethoxy, 2-fluoroethoxy, 2-chloroethoxy, 2,2-difluoroethoxy and 2,2,2-trichloroethoxy; preferably difluoromethoxy, 2-chloroethoxy and trifluoromethoxy.

Halophenyl is preferably phenyl substituted by 1, 2 or 3 halogen atoms, for example 4-chloro-phenyl. Halophenoxy is preferably phenoxy substituted by 1, 2 or 3 halogen atoms, for example 4-chloro-phenoxy. In the groups which represent the substituent G, the right free valence is bonded to the substituent B.

The compounds of formula I can occur in different isomeric forms; the invention covers all those isomers and mixtures thereof. The compounds of formula I, wherein R₃ is hydrogen, may occur in different tautomeric forms. For example, compounds of formula I exist in the tautomeric forms Ii and Iii:

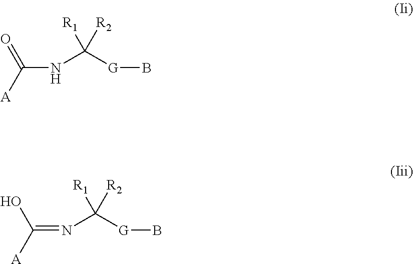

In a preferred group of compounds,

A is the group A₁

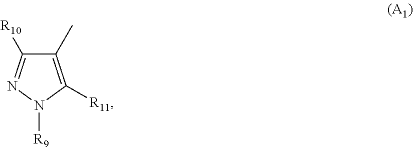

wherein
R₉ is methyl; R₁₀ is C₁-C₄haloalkyl, preferably difluoromethyl or trifluoromethyl and R₁₁ is hydrogen.

Preferred compounds of formula I are represented by formula Ia

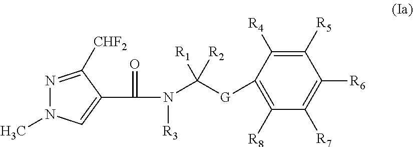

wherein R₃ is hydrogen and G, R₁, R₂, R₄, R₅, R₆, R₇ and R₈ are as defined for formula I. Preferred compounds of formula Ia wherein R₃ is hydrogen are listed in Table 1 below (compounds 1.001-1.260). Further preferred compounds of formula I are represented by formula Ia, wherein R₃ is cyclopropyl and the substituents G, R₁, R₂, R₄, R₅, R₆, R₇ and R₈ are as defined as for each of the individual compounds 1.001-1.260 listed in Table 1. The individual compounds of formula Ia, wherein R₃ is cyclopropyl are therefore defined as compounds 2.001-2.260. Further preferred compounds of formula I are those, in which R₁₀ is trifluoromethyl. From this group of compounds, the compounds represented by formula Ic

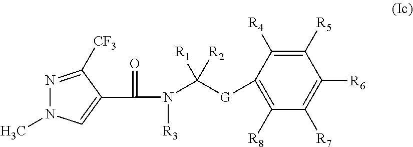

wherein R₃ is hydrogen and G, R₁, R₂, R₄, R₅, R₆, R₇ and R₈ are as defined under formula I are especially preferred. Individual compounds of this preferred group of compounds of formula Ic are described in Table 1 below, wherein R₃ is hydrogen and G, R₁, R₂, R₄, R₅, R₆, R₇ and R₈ are as defined in Table 1. Said compounds are therefore described as compounds 3.001-3.260. Further individual compounds of this preferred group of compounds of formula Ic are described in Table 1 below, wherein R₃ is cyclopropyl and G, R₁, R₂, R₄, R₅, R₆, R₇ and R₈ are as defined in Table 1. Said compounds are therefore described as compounds 4.001-4.260.

R₁ is preferably hydrogen. R₂ is preferably hydrogen or methyl. R₃ is preferably hydrogen. G is in particular selected from (CH₂)₂, (CH₂)₃ and CH₂OCH₂, R₄ is preferably halogen or methyl, in particular chlorine. R₅ is hydrogen or halogen, in particular hydrogen or chlorine. R₆ is preferably hydrogen or halogen, in particular chlorine. R₇ is preferably hydrogen or halogen, preferably hydrogen. R₈ is halogen, methyl or methoxy, in particular chlorine, methyl or methoxy most preferred chlorine.

Compounds of formula I may be prepared by reacting a compound of formula II

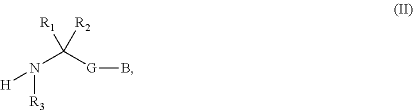

in which B, R₁, R₂, R₃ and G are as defined under formula I; with a compound of formula III

A-C(═O)—R* (III),

in which A is as defined under formula I, and R* is halogen, hydroxy or C₁-C₆ alkoxy, preferably chloro.

The reactions to give compounds of formula I are advantageously carried out in aprotic inert organic solvents. Such solvents are hydrocarbons such as benzene, toluene, xylene or cyclohexane, chlorinated hydrocarbons such as dichloromethane, trichloromethane, tetrachloromethane or chlorobenzene, ethers such as diethyl ether, ethylene glycol dimethyl ether, diethylene glycol dimethyl ether, tetrahydrofuran or dioxane, nitriles such as acetonitrile or propionitrile, amides such as N,N-dimethylformamide, diethylformamide or N-methylpyrrolidinone. The reaction temperatures are advantageously between −20° C. and +120° C. In general, the reactions are slightly exothermic and, as a rule, they can be carried out at ambient temperature. To shorten the reaction time, or else to start the reaction, the mixture may be heated briefly to the boiling point of the reaction mixture. The reaction times can also be shortened by adding a few drops of base as reaction catalyst. Suitable bases are, in particular, tertiary amines such as trimethylamine, triethylamine, quinuclidine, 1,4-diazabicyclo[2.2.2]octane, 1,5-diazabicyclo[4.3.0]non-5-ene or 1,5-diazabicyclo-[5.4.0]undec-7-ene. However, inorganic bases such as hydrides, e.g. sodium hydride or calcium hydride, hydroxides, e.g. sodium hydroxide or potassium hydroxide, carbonates such as sodium carbonate and potassium carbonate, or hydrogen carbonates such as potassium hydrogen carbonate and sodium hydrogen carbonate may also be used as bases. The bases can be used as such or else with catalytic amounts of a phase-transfer catalyst, for example a crown ether, in particular 18-crown-6, or a tetraalkylammonium salt.

Compounds of formula Ib

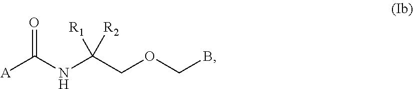

in which A, B, R₁ and R₂ are as defined under formula I and R₁ and R₂ independently of each other are hydrogen or C₁-C₄ alkyl; may be prepared as described in reaction scheme 1.

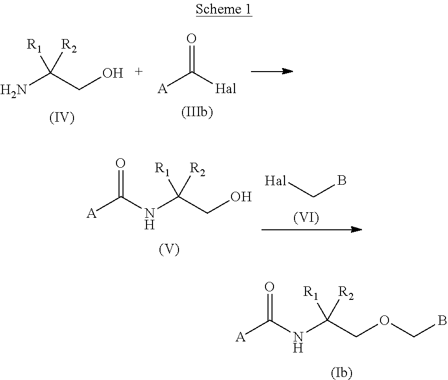

Compounds of formula V, in which A, R₁ and R₂ are as defined under formula Ib, may be prepared by reacting an amino alcohol of formula IV, in which R₁ and R₂ are as defined under formula Ib, with a compound of formula IIIb, in which A is as defined under formula Ib and Hal is halogen, preferably chloro, in the presence of a base, such as triethylamine, Hunig base, sodium bicarbonate, sodium carbonate, potassium carbonate, pyridine or quinoline, preferably triethylamine, and in a solvent, such as diethylether, TBME, THF, dichloromethane, chloroform, DMF or NMP, for between 10 minutes and 48 hours, preferably 12 to 24 hours, and at temperatures between 0° C. and reflux, preferably 20 to 25° C. Compounds of formula Ib may be prepared by the reaction of compounds of formula V and compounds of formula VI, in which B is as defined under formula Ib and Hal is halogen. This nucleophilic substitution reaction may preferably performed in the presence of a base, such as sodium carbonate, potassium carbonate or sodium hydride, but preferably sodium hydride, and in a solvent, such as THF, DMF, DMSO or NMP, for between 10 minutes and 48 hours, preferably 12 to 24 hours, and between 0° C. and reflux, preferably 60 to 100° C. The reaction of non-activated compounds of formula VI and compounds of formula V may be carried out under palladium- or copper-catalyst mediated conditions. Compounds of formula I, wherein R₃ is C₃-C₇cycloalkyl can be prepared analogously.

The compounds of the formula IV and VI, wherein the substituents as described above, are known and commercially available or can be prepared according to the above-mentioned references or according to methods known in the art.

The compounds of the formula IIIb, wherein the substituents as described above, are known and partially commercially available. They can be prepared analogously as described, for example, in WO 00/09482, WO 02/38542, WO 04/018438, EP-0-589-301, WO 93/11117 and Arch. Pharm. Res. 2000, 23(4), 315-323.

The intermediates of the formula Va

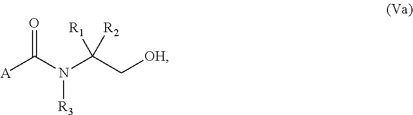

wherein A, R₁, R₂ and R₃ are as defined under formula I above are novel and are developed specifically for the preparation of the compounds of the formula Ib. Accordingly, these intermediates of the formula Va also form part of the subject-matter of the present invention.

Compounds of formula Ib1

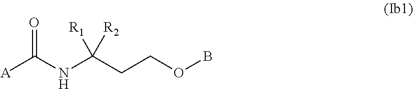

wherein A, B, R₁ and R₂ are as defined under formula I above; may be prepared as described in reaction scheme 2.

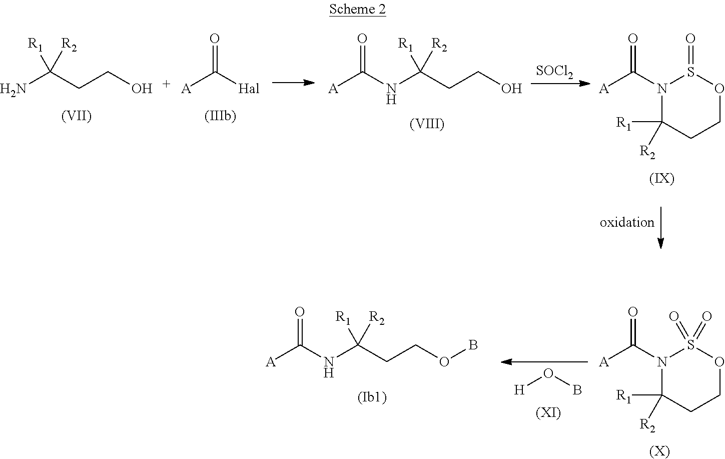

Compounds of formula VIII, in which A, R₁ and R₂ are as defined under formula Ib, may be prepared by reacting an amino alcohol of formula VII, in which R₁ and R₂ are as defined under formula Ib, with a compound of formula IIIb, in which A is as defined under formula Ib and Hal is halogen, preferably chloro, in the presence of a base, such as triethylamine, Hunig base, sodium bicarbonate, sodium carbonate, potassium carbonate, pyridine or quinoline, preferably triethylamine, and in a solvent, such as diethylether, TBME, THF, dichloromethane, chloroform, DMF or NMP, for between 10 minutes and 48 hours, preferably 12 to 24 hours, and at temperatures between 0° C. and reflux, preferably 20 to 25° C. Cyclic sulfamidites of formula IX, in which A, R₁ and R₂ are as defined under formula Ib, can then be prepared from the compounds of formula VIII by using SOCl₂. This cyclisation reaction may be performed in the presence of a base. A suitable base is pyridine. Suitable solvents include dichloromethane and nitriles such as acetonitrile and propionitrile. The reaction temperature is preferably in the range of −50° C. to 20° C.

Cyclic sulfamidates of formula X, in which A, R₁ and R₂ are as defined under formula Ib, may be prepared by oxidation of the cyclic sulfamidites of formula IX. Suitable oxidation reagents are RuO₄ and RuCl₃ 3H₂O in combination with NalO₄. Suitable solvents include mixtures of nitriles and water; as nitrile can be used, for example, acetonitrile or propionitrile. The reaction temperature is preferably in the range of 0° C. to 30° C.

For a review of preparation methods for cyclic sulfates and sulfamidates, see Lohray, B. B. in Advances in Heterocyclic Chemistry; Katritzky, A. R., Ed.; Academic Press: San Diego, 1997; Vol. 68, pp 89-180; and Posakony J. J., J. Org. Chem., 2002, 67, 5164-5169. The cyclic sulfamidates of formula X may then react with compounds of formula XI, in which B is as defined under formula Ib, to form compounds of formula Ib. This ring-opening by using oxygen nucleophiles may be performed in the presence of a base. Suitable bases include carbonates, such as lithium hydroxide, cesium carbonate, potassium carbonate, or metal hydrides, such as sodium hydride and lithium hydride. Suitable solvents include N,N-dimethylformamide, dimethylacetamide and DMSO. The reaction temperature can vary within wide limits, but typically is from ambient temperature to 100° C. Compounds of formula I, wherein R₃ is C₃-C₇cycloalkyl can be prepared analogously.

The compounds of the formula VII and XI, wherein the substituents as described above, are known and commercially available or can be prepared according to the above-mentioned references or according to methods known in the art.

The intermediates of formula VIIIa

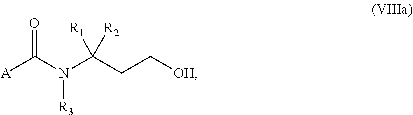

wherein A, R₁, R₂ and R₃ are as defined under formula I are novel and are developed specifically for the preparation of the compounds of the formula I b1. Accordingly, these intermediates of the formula VIIIa also form part of the subject-matter of the present invention.

The intermediates of formula XB

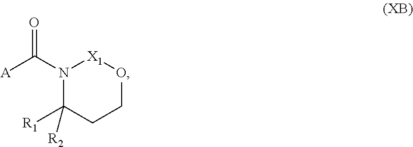

wherein A is as defined under formula I; X₁ is —S(O)— or —S(O)₂—; and R₁ and R₂ independently of each other stand for hydrogen or C₁-C₄ alkyl are novel and are developed specifically for the preparation of the compounds of the formula Ib1. Accordingly, these intermediates of the formula XB also form part of the subject-matter of the present invention.

Compounds of formula Ib2

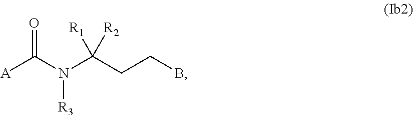

wherein A, B, R₁, R₂ and R₃ are as defined under formula I above may be prepared as described in reaction scheme 3.

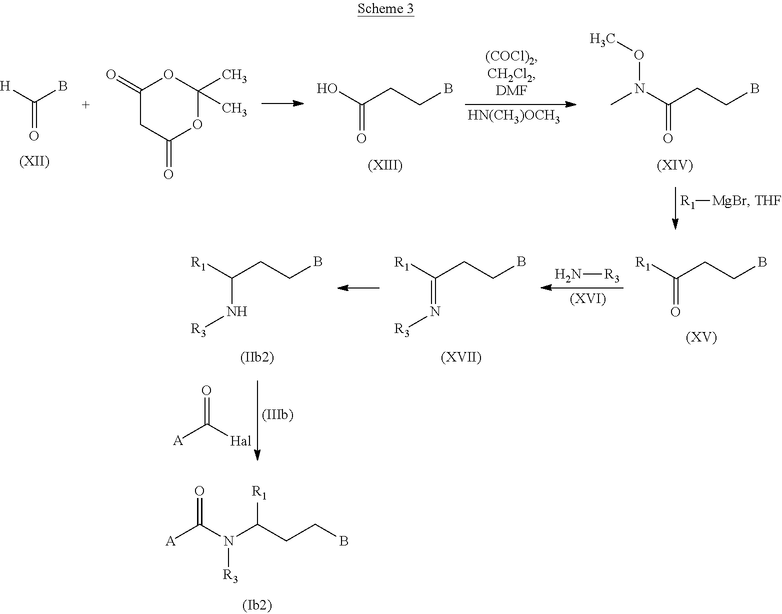

The acid of the formula XIII, prepared from the aldehyde of formula XII and Meldrum's acid wherein B is as defined under formula Ib, can be converted to the corresponding acylchloride and this acylchloride can then in situ be reacted with N,O-dimethylhydroxylamine to afford a Weinreb amide of formula XIV, wherein B is as defined under formula Ib. A subsequent reaction with a Grignard reagent of the formula R₁—MgBr, wherein R₁ is as defined under formula Ib, yields the ketone of formula XV.

Amines of formula IIb2 wherein R₁, R₃, and B are as defined under formula Ib may be prepared according to a process which comprises the reaction of a compound of general formula XV with an amine or an salt thereof of formula XVI to provide an imine derivative of general formula XVII. A second step comprises the reduction of the imine derivative of general formula XVII by hydrogenation or by an hydride donor, in the same or a different pot to provide an amine of general formula IIb2 or one of its salt. Preferably, the hydride donor is chosen as being metal or metal hydride such as LiAlH₄, NaBH₄, NaBH₃CN, NaBH(OAc)₃, KBH₄, B₂H₆.

The compounds of the formula XII, wherein the substituents as described above, are known and commercially available or can be prepared according to methods known in the art.

The intermediates of formula IIb2

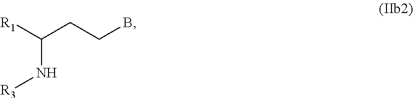

in which B, R₁ and R₃ is as defined under formula I are novel and are developed specifically for the preparation of the compounds of the formula Ib2. Accordingly, these intermediates of the formula IIb2 also form part of the subject-matter of the present invention.

Compounds of formula Ib3

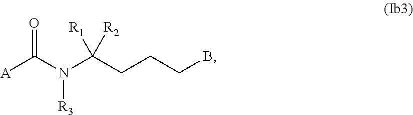

wherein A, B, R₁ and R₂ are as defined under formula I may be prepared as described in reaction scheme 4.

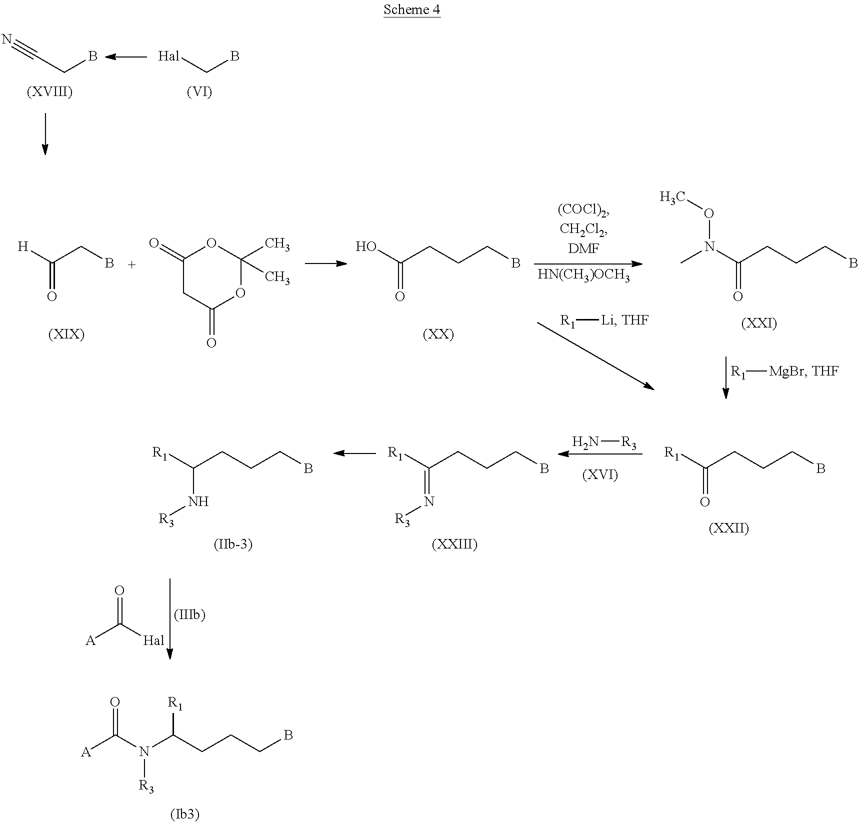

The aldehyde of formula XIX, prepared from the nitrile of formula XVIII, wherein B is defined under formula Ib3 can be converted with Meldrum's acid to the corresponding acid of formula XX by known methods. The acid of the formula XX wherein B is as defined under formula Ib3, can be converted to the corresponding acylchloride and this acylchloride can then in situ be reacted with N,O-dimethylhydroxylamine to afford a Weinreb amide of formula XXI, wherein B is as defined under formula Ib3. A subsequent reaction with a Grignard reagent of the formula R₁—MgBr, wherein R₁ is as defined under formula Ib3, yields the ketone of formula XXII. Alternatively the ketone XXII may be prepared by the reaction of the acid of formula XX with an alkyllithium reagent of the formula R₁—Li, wherein R₁ is as defined under formula Ib3.

Amines of formula IIb3 wherein R₁, R₃, and B are as defined under formula Ib may be prepared according to a process which comprises the reaction of a compound of general formula XXII with an amine or an salt thereof of formula XVI to provide an imine derivative of general formula XXIII. A second step comprises the reduction of the imine derivative of general formula XXIII by hydrogenation or by an hydride donor, in the same or a different pot to provide an amine of general formula IIb3 or one of its salt. Preferably, the hydride donor is chosen as being metal or metal hydride such as LiAlH₄, NaBH₄, NaBH₃CN, NaBH(OAc)₃, KBH₄, B₂H₆.

The intermediates of formula IIb3

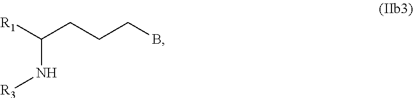

wherein B, R₁ and R₃ is as defined under formula I, are novel and are developed specifically for the preparation of the compounds of the formula Ib. Accordingly, said intermediates of formula IIb3 also form part of the subject-matter of the present invention.

The compounds of formula I can be isolated in the customary manner by concentrating and/or by evaporating the solvent and purified by recrystallization or trituration of the solid residue in solvents in which they are not readily soluble, such as ethers, aromatic hydrocarbons or chlorinated hydrocarbons.

The compounds I and, where appropriate, the tautomers thereof, can be present in the form of one of the isomers which are possible or as a mixture of these, for example in the form of pure isomers, such as antipodes and/or diastereomers, or as isomer mixtures, such as enantiomer mixtures, for example racemates, diastereomer mixtures or racemate mixtures, depending on the number, absolute and relative configuration of asymmetric carbon atoms which occur in the molecule and/or depending on the configuration of non-aromatic double bonds which occur in the molecule; the invention relates to the pure isomers and also to all isomer mixtures which are possible and is to be understood in each case in this sense hereinabove and hereinbelow, even when stereochemical details are not mentioned specifically in each case.

The compounds I and, where appropriate, the tautomers thereof, can, if appropriate, also be obtained in the form of hydrates and/or include other solvents, for example those which may have been used for the crystallization of compounds which are present in solid form.

It has now been found that the compounds of formula I according to the invention have, for practical purposes, a very advantageous spectrum of activities for protecting useful plants against diseases that are caused by phytopathogenic microorganisms, such as fungi, bacteria or viruses.

The invention relates to a method of controlling or preventing infestation of useful plants by phytopathogenic microorganisms, wherein a compound of formula I is applied as active ingredient to the plants, to parts thereof or the locus thereof. The compounds of formula I according to the invention are distinguished by excellent activity at low rates of application, by being well tolerated by plants and by being environmentally safe. They have very useful curative, preventive and systemic properties and are used for protecting numerous useful plants. The compounds of formula I can be used to inhibit or destroy the diseases that occur on plants or parts of plants (fruit, blossoms, leaves, stems, tubers, roots) of different crops of useful plants, while at the same time protecting also those parts of the plants that grow later e.g. from phytopathogenic microorganisms.

It is also possible to use compounds of formula I as dressing agents for the treatment of plant propagation material, in particular of seeds (fruit, tubers, grains) and plant cuttings (e.g. rice), for the protection against fungal infections as well as against phytopathogenic fungi occurring in the soil.

Furthermore the compounds of formula I according to the invention may be used for controlling fungi in related areas, for example in the protection of technical materials, including wood and wood related technical products, in food storage or in hygiene management.

The compounds of formula I are, for example, effective against the phytopathogenic fungi of the following classes: Fungi imperfecti (e.g. Botrytis, Pyricularia, Helminthosporium, Fusarium, Septoria, Cercospora and Alternaria) and Basidiomycetes (e.g. Rhizoctonia, Hemileia, Puccinia). Additionally, they are also effective against the Ascomycetes classes (e.g. Venturia and Erysiphe, Podosphaera, Monilinia, Uncinula) and of the Oomycetes classes (e.g. Phytophthora, Pythium, Plasmopara). Outstanding activity has been observed against powdery mildew (Erysiphe spp.). Furthermore, the novel compounds of formula I are effective against phytopathogenic bacteria and viruses (e.g. against Xanthomonas spp, Pseudomonas spp, Erwinia amylovora as well as against the tobacco mosaic virus). Good activity has been observed against Asian soybean rust (Phakopsora pachyrhizi).

Within the scope of the invention, useful plants to be protected typically comprise the following species of plants: cereal (wheat, barley, rye, oat, rice, maize, sorghum and related species); beet (sugar beet and fodder beet); pomes, drupes and soft fruit (apples, pears, plums, peaches, almonds, cherries, strawberries, raspberries and blackberries); leguminous plants (beans, lentils, peas, soybeans); oil plants (rape, mustard, poppy, olives, sunflowers, coconut, castor oil plants, cocoa beans, groundnuts); cucumber plants (pumpkins, cucumbers, melons); fibre plants (cotton, flax, hemp, jute); citrus fruit (oranges, lemons, grapefruit, mandarins); vegetables (spinach, lettuce, asparagus, cabbages, carrots, onions, tomatoes, potatoes, paprika); lauraceae (avocado, cinnamomum, camphor) or plants such as tobacco, nuts, coffee, eggplants, sugar cane, tea, pepper, vines, hops, bananas and natural rubber plants, as well as ornamentals.

The term “useful plants” is to be understood as including also useful plants that have been rendered tolerant to herbicides like bromoxynil or classes of herbicides (such as, for example, HPPD inhibitors, ALS inhibitors, for example primisulfuron, prosulfuron and trifloxysulfuron, EPSPS (5-enol-pyrovyl-shikimate-3-phosphate-synthase) inhibitors, GS (glutamine synthetase) inhibitors or PPO (protoporphyrinogen-oxidase) inhibitors) as a result of conventional methods of breeding or genetic engineering. An example of a crop that has been rendered tolerant to imidazolinones, e.g. imazamox, by conventional methods of breeding (mutagenesis) is Clearfield® summer rape (Canola). Examples of crops that have been rendered tolerant to herbicides or classes of herbicides by genetic engineering methods include glyphosate- and glufosinate-resistant maize varieties commercially available under the trade names RoundupReady®, Herculex I® and LibertyLink®.

The term “useful plants” is to be understood as including also useful plants which have been so transformed by the use of recombinant DNA techniques that they are capable of synthesising one or more selectively acting toxins, such as are known, for example, from toxin-producing bacteria, especially those of the genus Bacillus.

Examples of such plants are: YieldGard® (maize variety that expresses a CryIA(b) toxin); YieldGard Rootworm® (maize variety that expresses a CryIIIB(b1) toxin); YieldGard Plus® (maize variety that expresses a CryIA(b) and a CryIIIB(b1) toxin); Starlink® (maize variety that expresses a Cry9(c) toxin); Herculex I® (maize variety that expresses a CryIF(a2) toxin and the enzyme phosphinothricine N-acetyltransferase (PAT) to achieve tolerance to the herbicide glufosinate ammonium); NuCOTN 33B® (cotton variety that expresses a CryIA(c) toxin); Bollgard I® (cotton variety that expresses a CryIA(c) toxin); Bollgard II® (cotton variety that expresses a CryIA(c) and a CryIIA(b) toxin); VIPCOT® (cotton variety that expresses a VIP toxin); NewLeaf® (potato variety that expresses a CryIIIA toxin); Nature-Gard® Agrisure® GT Advantage (GA21 glyphosate-tolerant trait), Agrisure® CB Advantage (Bt11 corn borer (CB) trait), Agrisure® RW (corn rootworm trait) and Protecta®.

The term “useful plants” is to be understood as including also useful plants which have been so transformed by the use of recombinant DNA techniques that they are capable of synthesising antipathogenic substances having a selective action, such as, for example, the so-called “pathogenesis-related proteins” (PRPs, see e.g. EP-A-0 392 225). Examples of such antipathogenic substances and transgenic plants capable of synthesising such antipathogenic substances are known, for example, from EP-A-0 392 225, WO 95/33818, and EP-A-0 353 191. The methods of producing such transgenic plants are generally known to the person skilled in the art and are described, for example, in the publications mentioned above.

The term “locus” of a useful plant as used herein is intended to embrace the place on which the useful plants are growing, where the plant propagation materials of the useful plants are sown or where the plant propagation materials of the useful plants will be placed into the soil. An example for such a locus is a field, on which crop plants are growing.

The term “plant propagation material” is understood to denote generative parts of the plant, such as seeds, which can be used for the multiplication of the latter, and vegetative material, such as cuttings or tubers, for example potatoes. There may be mentioned for example seeds (in the strict sense), roots, fruits, tubers, bulbs, rhizomes and parts of plants. Germinated plants and young plants which are to be transplanted after germination or after emergence from the soil, may also be mentioned. These young plants may be protected before transplantation by a total or partial treatment by immersion. Preferably “plant propagation material” is understood to denote seeds.

The compounds of formula I can be used in unmodified form or, preferably, together with carriers and adjuvants conventionally employed in the art of formulation.

Therefore the invention also relates to compositions for controlling and protecting against phytopathogenic microorganisms, comprising a compound of formula I and an inert carrier, and to a method of controlling or preventing infestation of useful plants by phytopathogenic microorganisms, wherein a composition, comprising a compound of formula I as active ingredient and an inert carrier, is applied to the plants, to parts thereof or the locus thereof.

To this end compounds of formula I and inert carriers are conveniently formulated in known manner to emulsifiable concentrates, coatable pastes, directly sprayable or dilutable solutions, dilute emulsions, wettable powders, soluble powders, dusts, granulates, and also encapsulations e.g. in polymeric substances. As with the type of the compositions, the methods of application, such as spraying, atomising, dusting, scattering, coating or pouring, are chosen in accordance with the intended objectives and the prevailing circumstances. The compositions may also contain further adjuvants such as stabilizers, antifoams, viscosity regulators, binders or tackifiers as well as fertilizers, micronutrient donors or other formulations for obtaining special effects.

Suitable carriers and adjuvants can be solid or liquid and are substances useful in formulation technology, e.g. natural or regenerated mineral substances, solvents, dispersants, wetting agents, tackifiers, thickeners, binders or fertilizers. Such carriers are for example described in WO 97/33890.

The compounds of formula I or compositions, comprising a compound of formula I as active ingredient and an inert carrier, can be applied to the locus of the plant or plant to be treated, simultaneously or in succession with further compounds. These further compounds can be e.g. fertilizers or micronutrient donors or other preparations which influence the growth of plants. They can also be selective herbicides as well as insecticides, fungicides, bactericides, nematicides, molluscicides or mixtures of several of these preparations, if desired together with further carriers, surfactants or application promoting adjuvants customarily employed in the art of formulation.

A preferred method of applying a compound of formula I, or a composition, comprising a compound of formula I as active ingredient and an inert carrier, is foliar application. The frequency of application and the rate of application will depend on the risk of infestation by the corresponding pathogen. However, the compounds of formula I can also penetrate the plant through the roots via the soil (systemic action) by drenching the locus of the plant with a liquid formulation, or by applying the compounds in solid form to the soil, e.g. in granular form (soil application). In crops of water rice such granulates can be applied to the flooded rice field. The compounds of formula I may also be applied to seeds (coating) by impregnating the seeds or tubers either with a liquid formulation of the fungicide or coating them with a solid formulation.

A formulation, i.e. a composition comprising the compound of formula I and, if desired, a solid or liquid adjuvant, is prepared in a known manner, typically by intimately mixing and/or grinding the compound with extenders, for example solvents, solid carriers and, optionally, surface-active compounds (surfactants).

The agrochemical formulations will usually contain from 0.1 to 99% by weight, preferably from 0.1 to 95% by weight, of the compound of formula I, 99.9 to 1% by weight, preferably 99.8 to 5% by weight, of a solid or liquid adjuvant, and from 0 to 25% by weight, preferably from 0.1 to 25% by weight, of a surfactant.

Whereas it is preferred to formulate commercial products as concentrates, the end user will normally use dilute formulations.

Advantageous rates of application are normally from 5 g to 2 kg of active ingredient (a.i.) per hectare (ha), preferably from 10 g to 1 kg a.i./ha, most preferably from 20 g to 600 g a.i./ha. When used as seed drenching agent, convenient rates of application are from 10 mg to 1 g of active substance per kg of seeds. The rate of application for the desired action can be determined by experiments. It depends for example on the type of action, the developmental stage of the useful plant, and on the application (location, timing, application method) and can, owing to these parameters, vary within wide limits.

Surprisingly, it has now been found that the compounds of formula I can also be used in methods of protecting crops of useful plants against attack by phytopathogenic organisms as well as the treatment of crops of useful plants infested by phytopathogenic organisms comprising administering a combination of glyphosate and at least one compound of formula I to the plant or locus thereof, wherein the plant is resistant or sensitive to glyphosate.

Said methods may provide unexpectedly improved control of diseases compared to using the compounds of formula I in the absence of glyphosate. Said methods may be effective at enhancing the control of disease by compounds of formula I. While the mixture of glyphosate and at least one compound of formula I may increase the disease spectrum controlled, at least in part, by the compound of formula I, an increase in the activity of the compound of formula I on disease species already known to be controlled to some degree by the compound of formula I can also be the effect observed.

Said methods are particularly effective against the phytopathogenic organisms of the kingdom Fungi, phylum Basidiomycot, class Uredinomycetes, subclass Urediniomycetidae and the order Uredinales (commonly referred to as rusts). Species of rusts having a particularly large impact on agriculture include those of the family Phakopsoraceae, particularly those of the genus Phakopsora, for example Phakopsora pachyrhizi, which is also referred to as Asian soybean rust, and those of the family Pucciniaceae, particularly those of the genus Puccinia such as Puccinia graminis, also known as stem rust or black rust, which is a problem disease in cereal crops and Puccinia recondita, also known as brown rust.

An embodiment of said method is a method of protecting crops of useful plants against attack by a phytopathogenic organism and/or the treatment of crops of useful plants infested by a phytopathogenic organism, said method comprising simultaneously applying glyphosate, including salts or esters thereof, and at least one compound of formula I, which has activity against the phytopathogenic organism to at least one member selected from the group consisting of the plant, a part of the plant and the locus of the plant.

The compounds of formula (I), or a pharmaceutical salt thereof, described above may also have an advantageous spectrum of activity for the treatment and/or prevention of microbial infection in an animal. “Animal” can be any animal, for example, insect, mammal, reptile, fish, amphibian, preferably mammal, most preferably human. “Treatment” means the use on an animal which has microbial infection in order to reduce or slow or stop the increase or spread of the infection, or to reduce the infection or to cure the infection. “Prevention” means the use on an animal which has no apparent signs of microbial infection in order to prevent any future infection, or to reduce or slow the increase or spread of any future infection.

According to the present invention there is provided the use of a compound of formula (I) in the manufacture of a medicament for use in the treatment and/or prevention of microbial infection in an animal. There is also provided the use of a compound of formula (I) as a pharmaceutical agent. There is also provided the use of a compound of formula (I) as an antimicrobial agent in the treatment of an animal. According to the present invention there is also provided a pharmaceutical composition comprising as an active ingredient a compound of formula (I), or a pharmaceutically acceptable salt thereof, and a pharmaceutically acceptable diluent or carrier. This composition can be used for the treatment and/or prevention of antimicrobial infection in an animal. This pharmaceutical composition can be in a form suitable for oral administration, such as tablet, lozenges, hard capsules, aqueous suspensions, oily suspensions, emulsions dispersible powders, dispersible granules, syrups and elixirs. Alternatively this pharmaceutical composition can be in a form suitable for topical application, such as a spray, a cream or lotion. Alternatively this pharmaceutical composition can be in a form suitable for parenteral administration, for example injection. Alternatively this pharmaceutical composition can be in inhalable form, such as an aerosol spray.

The compounds of formula (I) may be effective against various microbial species able to cause a microbial infection in an animal. Examples of such microbial species are those causing Aspergillosis such as Aspergillus fumigatus, A. flavus, A. terrus, A. nidulans and A. niger, those causing Blastomycosis such as Blastomyces dermatitidis; those causing Candidiasis such as Candida albicans, C. glabrata, C. tropicalis, C. parapsilosis, C. krusei and C. lusitaniae; those causing Coccidioidomycosis such as Coccidioides immitis; those causing Cryptococcosis such as Cryptococcus neoformans; those causing Histoplasmosis such as Histoplasma capsulatum and those causing Zygomycosis such as Absidia corymbifera, Rhizomucor pusillus and Rhizopus arrhizus. Further examples are Fusarium Spp such as Fusarium oxysporum and Fusarium solani and Scedosporium Spp such as Scedosporium apiospermum and Scedosporium prolificans. Still further examples are Microsporum Spp, Trichophyton Spp, Epidermophyton Spp, Mucor Spp, Sporothorix Spp, Phialophora Spp, Cladosporium Spp, Petriellidium spp, Paracoccidioides Spp and Histoplasma Spp.

The following non-limiting Examples illustrate the above-described invention in greater detail without limiting it.

### PREPARATION EXAMPLES

### Example P1

### Preparation of 3-difluoromethyl-1-methyl-1H-pyrazole-4-carboxylic acid [1-methyl-3-(2,4,6-trichloro-phenyl)-propyl]-amide (compound 1.004)

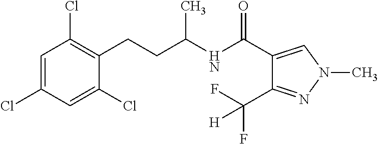

To the suspension of 3-difluoromethyl-1-methyl-1H-pyrazole-4-carboxylic acid (190 mg, 1.08 mmol) in 10 ml dichloromethane under nitrogen environment few drops of DMF is added to make a clear solution. HOBT (248 mg, 1.62 mmol) is added at ambient temperature and after 20 mins EDCI (310 mg, 1.62 mmol) is added to the above solution maintained at 0-5° C. After 30 minutes 1-methyl-3-(2,4,6-trichloro-phenyl)-propylamine (300 mg, 1.19 mmol), which is prepared as described in example P5d, is added to the solution at ambient temperature followed by catalytic amount of DMAP. Reaction mixture is kept at ambient temperature for 3 hours for completion. Reaction mixture is diluted with water (15 ml) and aq. layer is extracted with dichloromethane (3×30 ml). Combined organic layer is washed with HCl (10 ml, 1N) followed by saturated NaHCO₃ (10 ml), water (20 ml) and brine (15 ml). Organic layer is dried over sodium sulfate and concentrated under vacuum to give brownish mass. Crude product is purified by silica-gel chromatography.

0.14 g (50% of theory) of 3-difluoromethyl-1-methyl-1H-pyrazole-4-carboxylic acid [2-(4-acetyl-p-phenoxy)-1-methyl-ethyl]-amide (compound 1.004) is obtained in form of a white solid (m.p. 100° C.).

¹H NMR: (CDCl3, 400 MHz):

7.93 (s, 1H); 7.32 (s, 1H); 6.94-6.67 (t, J=54, 1H); 6.26 (s, 1H); 4.31-4.24 (m, 1H); 3.92 (s, 3H); 2.99-2.92 (m, 2H); 1.79-1.67 (m, 2H); 1.29-1.27 (d, J=6.4, 3H)

MS [M+H]⁺ 410/412/414.

### Example P2

### Preparation of 3-difluoromethyl-1-methyl-1H-pyrazole-4-carboxylic acid [1-methyl-4-(2,4,6-trichloro-phenyl)-butyl]-amide (compound 1.069)

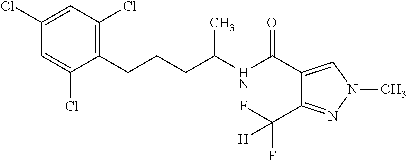

To a stirred solution of 1-methyl-4-(2,4,6-trichloro-phenyl)-butylamine (70 mg, 0.26 mmol), which is prepared as described in example P6f, in pyridine (0.8 ml) kept under nitrogen atmosphere, difluoromethyl-1-methyl-1H-pyrazole-4-carboxylic acid (42 mg, 0.23 mmol) is added at ambient temperature. Phosphoroxy chloride (0.024 ml, 0.26 mmol) is added dropwise at 0-5° C. Reaction mixture is refluxed for 4 hrs at 80° C. to complete the conversion. Reaction mixture is diluted with 15 ml of water and the product is extracted with ethyl acetate (3×25 ml). The combined organic layer is washed with brine (15 ml) and dried over sodium sulfate and it is concentrated under vacuum to give brownish solid crude, which is purified by silica-gel chromatography to afford the desired final product as off-white solid (35 mg, 31%). 0.035 g (31% of theory) of 3-difluoromethyl-1-methyl-1H-pyrazole-4-carboxylic acid [1-methyl-4-(2,4,6-trichloro-phenyl)-butyl]amide (compound 1.069) is obtained in form of a white solid (m.p. 120° C.).

¹H NMR: (CDCl3, 400 MHz):

7.89 (s, 1H); 7.27 (s, 2H); 6.92-6.65 (m, 1H); 6.14 (s, 1H); 4.22-4.14 (m, 1H); 3.91 (s, 3H); 2.90-2.86 (m, 2H); 1.62-1.60 (m, 4H); 1.20 (d, J=6.8 Hz, 3H).

MS [M+H]⁺ 424/426/428.

### Example P3

### Preparation of 3-difluoromethyl-1-methyl-1H-pyrazole-4-carboxylic acid [2-(2,6-dichloro-benzyloxy)-1-methyl-ethyl]-amide (compound 1.197)

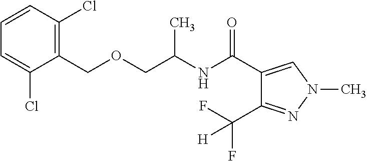

To a suspension of sodium hydride 50% in oil (10 mg; 0.39 mmol) in dimethylformamide (3 ml) sodium hydride 50% in oil (0.12 g; 2.5 mmol) is added 3-difluoromethyl-1-methyl-1H-pyrazole-4-carboxylic acid (2-hydroxy-1-methyl-ethyl)-amide (90 mg; 0.39 mmol), prepared as described in example P4 via syringe at ambient temperature. The reaction mixture is stirred for 20 minutes at ambient temperature followed by the addition of 1,3-dichloro-2-chloromethyl-benzene (78 mg; 0.40 mmol). The reaction mixture is stirred for 15 hours at ambient temperature then poured onto 1M HCl (20 ml) and extracted with ethyl acetate (2×20 ml). The combined ethyl acetate layers are washed with water (20 ml), brine (20 ml) and then dried over Na₂SO₄. After removal of the solvent the residue (152 mg oil) is purified by flash chromatography twice over silica gel (eluent: cyclo hexane/ethyl acetate 1:9 followed by eluent dichloromethane/methanol 19:1). 20 mg (13.1% of theory) of 3-difluoromethyl-1-methyl-1H-pyrazole-4-carboxylic acid [2-(2,6-dichloro-benzyloxy)-1-methyl-ethyl]-amide (compound 1.197) is obtained in form of an oil.

¹H NMR (400 MHz, CDCl₃): δ 1.37-1.39 (d, 3H, CH₃), 3.52-3.58 (m, 2H, CH₂),

3.91 (s, 3H, CH₃), 4.32-4.41 (m, 1H, CH), 4.59 (s, 2H, CH₂), 6.57 (s, 1H, NH),

6.71-6.97 (t, 1H, CHF2), 7.23 (d×d, 1H, Ar—H), 7.36 (d, 1H, Ar—H), 7.42 (d, 1H, Ar—H), 7.88 (t, 1H, Py-H).

MS [M+H]⁺ 392/394/396.

### Example P4

### Preparation of 3-difluoromethyl-1-methyl-1H-pyrazole-4-carboxylic acid (2-hydroxy-1-methyl-ethyl)-amide

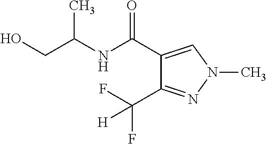

At a temperature of 0° C., a solution of 38.9 g 3-difluoromethyl-1-methyl-1H-pyrazole-4-carbonyl chloride (0.2 mol) in 100 ml dichloromethane is added dropwise to a stirred solution of 15 g alaminol (0.2 mol) and 25 g triethylamine (0.25 mol) in 400 ml dichloromethane. The reaction mixture is stirred for 1 h at ambient temperature and then allowed to stand for 3 h at ambient temperature. After removal of the solvent the residue is purified by flash chromatography over silica gel 400 g (eluent: ethyl acetate/methanol 19:1). 42 g (90% of theory) of 3-difluoromethyl-1-methyl-1H-pyrazole-4-carboxylic acid (2-hydroxy-1-methyl-ethyl)-amide is obtained in form of a solid (mp. 81-87° C.).

¹H NMR (400 MHz, CDCl₃): δ 1.23-1.26 (d, 3H), 2.97 (s, 1H₂OH), 3.57-3.73 (ddd, 2H),

3.94 (s, 3H), 4.17-4.23 (m, 1H), 6.57 (s, 1H), 6.75-7.02 (t, 1H), 7.90 (s, 1H).

MS [M+H]⁺ 234.

### Example P5

### Preparation of 1-methyl-3-(2,4,6-trichloro-phenyl)-propylamine

### a) Preparation of 3-(2,4,6-trichloro-phenyl)-propionic acid

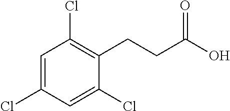

Meldrum's acid (2.06 g, 14.32 mmoles) is added to TEAF (7.05 ml) at ambient temperature under stirring and the reaction mixture is kept under nitrogen atmosphere. After 10 min 2,4,6-trichloro benzaldehyde (3 g, 14.32 mmoles) is added to the reaction mixture. It is then refluxed at 120° C. for 3 hrs. On completion of the reaction, the mixture is cooled at ambient and is poured into ice water (50 ml). Then aq. layer is extracted with ethyl acetate (3×80 ml), the combined organic layer is washed with brine (40 ml) and dried over sodium sulphate. Organic layer is concentrated under vacuum. 3.10 g (86.0% of theory) of 3-(2,4,6-trichloro-phenyl)-propionic acid is obtained in form of a white solid.

¹H NMR (400 MHz, CDCl3):

7.32 (s, 2H); 3.26-3.22 (m, 2H); 2.63-2.58 (m, 2H).

MS [M+H]⁺ 253/255/257

TEAF (triethyl ammonium formate) preparation: To formic acid (1.12 g˜0.93 ml, 24.5 mmoles) solution kept under N2 is added slowly triethylamine (1 g˜1.37 ml, 9.8 mmoles) at 0° C. and the mixture is stirred for one and half hour at AMBIENT TEMPERATURE.

### b) Preparation of N-methoxy-N-methyl-3-(2,4,6-trichloro-phenyl)-propionamide

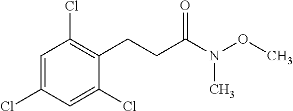

To a solution of acid chloride in chloroform [acid chloride is prepared from corresponding 3-(2,4,6-trichloro-phenyl)-propionic acid (5 g, 19.7 mmoles) using thionyl chloride (5.87 g, 49.4 mmole) under refluxing condition at 110° C. for 3 hours] N,O-dimethylhydroxylamine hydrochloride (2.3 g, 23.9 mmoles) followed by pyridine (3.77 ml, 47.7 mmole) is added drop wise at 0° C. The reaction mix is stirred at ambient temperature for 3 hours. On completion, the reaction mix is diluted with 70 ml DCM and 40 ml of water. Organic layer is separated and the aqueous layer is extracted two times with DCM (50 ml×2). The combined organic layer is washed with brine (40 ml) and dried over sodium sulfate. After removal of the solvent the residue is purified by chromatography over silica gel. 3.0 g (52.0% of theory) of N-methoxy-N-methyl-3-(2,4,6-trichloro-phenyl)-propionamide is obtained in form of a solid.

¹H NMR (CDCl3, 400 MHz):

7.31 (s, 2H); 3.67 (s, 3H); 3.24-3.19 (m, 2H); 3.19 (s, 3H); 2.67-2.63 (m, 2H)

MS [M+H]⁺ 296/298/300

### c) Preparation of 4-(2,4,6-trichloro-phenyl)-butan-2-one

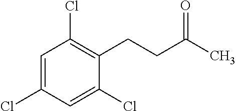

To the stirred and cold solution (0° C.) of N-methoxy-N-methyl-3-(2,4,6-trichloro-phenyl)-propionamide (1 g, 3.37 mmol) in THF (15 ml) kept under N2 atmosphere, MeMgl (1.3 mL of 3M ether solution, 3.9 mmole) is added and stirring is continued at 0° C. for 2 hours. Reaction mixture is quenched with 5% of HCl (10 ml) at 0° C. and aqueous layer is extracted with EtOAc (3×30 ml). The combined organic layer is washed with 20 ml of brine and dried over sodium sulfate; concentrated under vacuum to give off-white solid. The residue is purified by chromatography over silica gel. 0.50 g (60.0% of theory) of 4-(2,4,6-trichloro-phenyl)-butan-2-one is obtained in form of a solid.

¹H NMR (CDCl3, 400 MHz):

7.30 (s, 2H); 3.16-3.12 (m, 2H); 2.67-2.63 (m, 2H); 2.19 (s, 3H)

MS [M+H]⁺ 252/254/256

### d) Preparation of 1-methyl-3-(2,4,6-trichloro-phenyl)-propylamine (compound Z1.004)

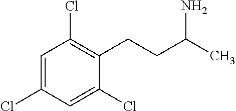

To a stirred solution of 4-(2,4,6-trichloro-phenyl)-butan-2-one (500 mg, 1.99 mmol) in methanol (10 ml), ammonium acetate (1.83 g, 23.88 mmol) is added at ambient temperature. After 30 minutes sodium cyanoborohydride (0.626 g, 9.95 mmol) is added under nitrogen environment at 0° C. and the mixture is stirred for 5 hrs. Reaction is monitored by TLC (2:3 ethyl acetate:hexane) and then quenched with 20 ml of 2N HCl. Aqueous layer is washed with 10 ml of ether and then basified to pH 12 and saturated with NaCl. Then aq. layer is extracted with DCM (3×40 ml). and the combined organic layer is dried over sodium sulfate and concentrated under vacuum. 0.35 g (70.0% of theory) of 1-methyl-3-(2,4,6-trichloro-phenyl)-propylamine (compound Z1.004) is obtained in form of a brownis liquid.

¹H NMR: (CDCl3, 400 MHz):

7.29 (s, 2H); 3.01-2.85 (m, 3H); 1.61-1.47 (m, 2H); 1.15 (d, J=6.4, 2H)

MS [M+H]⁺ 254/256/258

### Example P6

### 1-methyl-4-(2,4,6-trichloro-phenyl)-butylamine

### a) Preparation of 1,3,5-trichloro-2-chloromethyl-benzene

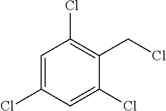

To a stirred solution of 2,4,6-trichlorobenzylalcohol (10.0 g; 47.3 mmoles) in chloroform (100 ml) kept under nitrogen atmosphere, thionyl chloride (6.07 mL, 85.1 mmole) is added slowly at 0° C. over a period of 15 minutes followed by catalytic amount of DMF. The reaction mix is allowed to stir at ambient temp for 3 hr. The reaction mixture is quenched with 50 mL of water; the aqueous layer is extracted with DCM (3×100 ml). The combined organic layer is washed with 5% Sodium bicarbonate solution (2×50 ml) followed by brine (50 ml) and dried over anhydrous sodium sulphate. The solvent is evaporated under reduced pressure. 10.9 g (100.0% of theory) of 1,3,5-trichloro-2-chloromethyl-benzene is obtained in form of a white solid.

¹HNMR (CDCl3, 400 MHz): δ 7.37 (2H, s); 4.82 (2H, s)

Mass: M=229.8

### b) Preparation of (2,4,6-trichloro-phenyl)-acetonitrile

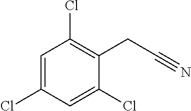

To a stirred solution of 1,3,5-trichloro-2-chloromethyl-benzene (12.85 g, 58.8 mmole) in ethanol (45 ml), NaCN (3.20, 67.0 mmole) solution in water (15 ml) is added at ambient temperature. The reaction mixture is refluxed for 4 hours to complete the reaction. Ethanol is evaporated and the reaction mixture is diluted with water (50 ml). The aqueous layer is extracted with EtOAc (3×100 ml). The combined organic layer is washed with brine (30 ml) and dried over sodium sulfate. Organic layer is concentrated under vacuum to give white solid. Crude product is purified by chromatography on silica-gel column to afford 9.0 g (75.0% of theory) of (2,4,6-trichloro-phenyl)-acetonitrile

¹HNMR (CDCl3, 400 MHz): 7.41 (s, 2H); 3.97 (s, 2H).

Mass: M=218.9

### c) Preparation of (2,4,6-trichloro-phenyl)-acetaldehyde

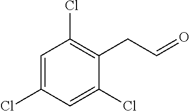

To the stirred, cold solution (−70° C.) of (2,4,6-trichloro-phenyl)-acetonitrile (5.7 g, 26.0 mmole) in toluene (120 ml) kept under N2 atmosphere, DIBAL-H (28.1 ml 1M solution in THF, 28.6 mmole) is added drop wise over a period of 30 min. Reaction is stirred for 3 hrs at −70° C. After completion of reaction, it is quenched by dropwise addition of HCl (60 ml, 2N) at −40° C. and is kept for 30 min at ambient temperature. Toluene layer is separated and aq. layer is extracted with ethyl acetate (3×100 ml) and. The combined organic layer is washed with brine (30 ml) and dried over sodium sulfate. The organic layer is concentrated under vacuum. 4.5 g (78.0% of theory) of (2,4,6-trichloro-phenyl)-acetaldehyde is obtained in form of a white solid.

¹HNMR (CDCl3, 400 MHz: 9.70 (s, 1H); 7.38 (s, 2H); 4.08 (s, 2H)

Mass: M=246.9

### d) Preparation of 4-(2,4,6-trichloro-phenyl)-butyric acid

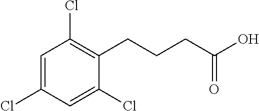

To the stirred solution of TEAF (2.25 ml) at ambient temperature is added Meldrum's acid (0.96 g, 6.70 mmoles) and the reaction is kept under nitrogen atmosphere. After 10 minutes (2,4,6-trichloro-phenyl)-acetaldehyde (1.5 g, 6.70 mmoles) is added to the reaction mixture. After 20 min. the reaction mixture is refluxed at 120° C. for 3 hours. On completion of the reaction, the mixture is cooled to ambient temperature and it is poured into ice water (20 ml). The product is extracted with EtOAc (3×50 ml); the combined organic layer is washed with brine (20 ml) and dried over sodium sulfate. Organic layer is concentrated under vacuum. The brown solid is purified through silica gel chromatography. 0.57 g (40.0% of theory) of 4-(2,4,6-trichloro-phenyl)-butyric acid is obtained in form of a white solid.

¹H NMR: (400M Hz in CDCl3): 10.03 (s, 1H), 7.32 (s, 2H); 2.97-2.93 (m, 2H); 2.45 (t, J=7.6 Hz, 2H); 1.95-1.88 (m, 2H)

Mass: M−1=264.93

TEAF preparation: To formic acid (1.12 g˜0.93 ml, 24.5 mmoles), kept under N2, is added slowly triethylamine (1 g˜1.37 ml, 9.8 mmoles) at 0° C. and the mixture is stirred for one and half hour at ambient temperature.

### e) Preparation of 5-(2,4,6-trichloro-phenyl)-pentan-2-one

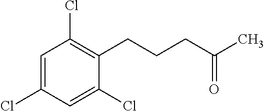

To a stirred and cold (0° C.) solution of 4-(2,4,6-trichloro-phenyl)-butyric acid (570 mg, 2.31 mmole) in water free diethyl ether (8 ml) kept under N2 atmosphere, Methyl lithium solution in ether (1.6 ml 3M ethereal solution, 4.82 mmole) is added dropwise during 15 minutes. Reaction mixture is stirred at ambient temperature for 3 hours. It is then quenched with 20 ml of water and the aqueous layer is saturated with NaCl and the product is extracted with EtOAc (3×40 ml). The combined organic layer is dried over sodium sulfate and is concentrated under vacuum to give yellow liquid. The crude is purified by silica-gel chromatography. 90 mg (17.0% of theory) of 5-(2,4,6-trichloro-phenyl)-pentan-2-one is obtained in form of a yellow solid.

¹H NMR: (400 M Hz in CDCl3): 7.29 (s, 2H); 2.9-2.8 (m, 2H); 2.52-2.49 (m, 2H); 2.15 (s, 3H); 1.89-1.82 (m, 2H)

Mass: M=263.9

### f) Preparation of 1-methyl-4-(2,4,6-trichloro-phenyl)-butylamine (compound Z1.069)

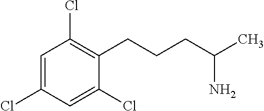

To a stirred solution of 5-(2,4,6-trichloro-phenyl)-pentan-2-one (170 mg, 0.64 mmol) in methanol (4 ml), kept under N2 atmosphere, ammonium acetate (600 mg, 7.68 mmol) is added at ambient temperature. After 30 min sodium cyanoborohydride (200 mg, 3.20 mmole) is added under nitrogen environment at 0° C. The reaction mixture is stirred further at ambient temperature for 5 hours, and then it is quenched with HCl (10 ml, 2N aq. solution). The aqueous layer is washed with ether (5 ml) and is then basified to pH 12 and saturated with NaCl. The product is extracted with DCM (3×30 ml). Combined organic layer is dried over sodium sulfate and concentrated under vacuum. 70 mg (41.0% of theory) of 1-methyl-4-(2,4,6-trichloro-phenyl)-butylamine is obtained in form of a brownish liquid.

¹H NMR: (400 M Hz in CDCl3): 7.29 (s, 2H); 3.41-3.31 (m, 1H); 2.92-2.87 (m, 2H); 1.70-1.64 (m, 4H); 1.43 (d, J=6.4, 3H)

TABLE 1
compounds of formula Ia:
(Ia)

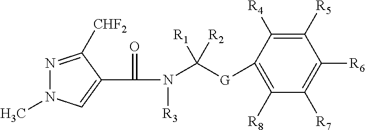

| Cpd | R₁ | R₂ | G | R₄ | R₅ | R₆ | R₇ | R₈ |
|---|---|---|---|---|---|---|---|---|
| 1.001 | H | Me | (CH₂)₂ | Cl | H | Cl | H | H |
| 1.002 | H | Me | (CH₂)₂ | Cl | H | H | H | Cl |
| 1.003 | H | Me | (CH₂)₂ | Cl | H | Cl | Cl | H |
| 1.004 | H | Me | (CH₂)₂ | Cl | H | Cl | H | Cl |
| 1.005 | H | Me | (CH₂)₂ | Cl | H | Br | H | Cl |
| 1.006 | H | Me | (CH₂)₂ | Cl | H | I | H | Cl |
| 1.007 | H | Me | (CH₂)₂ | Cl | H | CHF₂ | H | Cl |
| 1.008 | H | Me | (CH₂)₂ | Cl | H | CF₃ | H | Cl |
| 1.009 | H | Me | (CH₂)₂ | Cl | H | C≡C—H | H | Cl |
| 1.010 | H | Me | (CH₂)₂ | Cl | H | C≡C—Me | H | Cl |
| 1.011 | H | Me | (CH₂)₂ | Cl | H | C≡C—t-Bu | H | Cl |
| 1.012 | H | Me | (CH₂)₂ | Cl | H | C≡C-cyclopropyl | H | Cl |
| 1.013 | H | Me | (CH₂)₂ | Cl | H | C≡C-p-Cl-phenyl | H | Cl |
| 1.014 | H | Me | (CH₂)₂ | Cl | H | CH═NOMe | H | Cl |
| 1.015 | H | Me | (CH₂)₂ | Cl | H | CH═NOEt | H | Cl |
| 1.016 | H | Me | (CH₂)₂ | Cl | H | CMe═NOMe | H | Cl |
| 1.017 | H | Me | (CH₂)₂ | Cl | H | OCF₃ | H | Cl |
| 1.018 | H | Me | (CH₂)₂ | Cl | H | OCH₂CH═CHCl₂ | H | Cl |
| 1.019 | H | Me | (CH₂)₂ | Cl | H | p-Cl-phenoxy | H | Cl |
| 1.020 | H | Me | (CH₂)₂ | Cl | H | Cl | Cl | H |
| 1.021 | H | Me | (CH₂)₂ | Cl | Cl | Cl | H | Cl |
| 1.022 | H | Me | (CH₂)₂ | Cl | Cl | Cl | Cl | Cl |
| 1.023 | H | Me | (CH₂)₂ | Cl | H | Cl | H | Me |
| 1.024 | H | Me | (CH₂)₂ | Cl | H | Br | H | Me |
| 1.025 | H | Me | (CH₂)₂ | Cl | H | I | H | Me |
| 1.026 | H | Me | (CH₂)₂ | Cl | H | CF₃ | H | Me |
| 1.027 | H | Me | (CH₂)₂ | Cl | H | C≡C—H | H | Me |
| 1.028 | H | Me | (CH₂)₂ | Cl | H | C≡C—t-Bu | H | Me |
| 1.029 | H | Me | (CH₂)₂ | Cl | H | CH═NOMe | H | Me |
| 1.030 | H | Me | (CH₂)₂ | Cl | H | OCF₃ | H | Me |
| 1.031 | H | Me | (CH₂)₂ | Cl | H | p-Cl-phenoxy | H | Me |
| 1.032 | H | Me | (CH₂)₂ | Cl | H | Cl | H | OMe |
| 1.033 | H | Me | (CH₂)₂ | Cl | H | Br | H | OMe |
| 1.034 | H | Me | (CH₂)₂ | Cl | H | I | H | OMe |
| 1.035 | H | Me | (CH₂)₂ | Cl | H | CF₃ | H | OMe |
| 1.036 | H | Me | (CH₂)₂ | Cl | H | C≡C—H | H | OMe |
| 1.037 | H | Me | (CH₂)₂ | Cl | H | C≡C—t-Bu | H | OMe |
| 1.038 | H | Me | (CH₂)₂ | Cl | H | CH═NOMe | H | OMe |
| 1.039 | H | Me | (CH₂)₂ | Cl | H | OCF₃ | H | OMe |
| 1.040 | H | Me | (CH₂)₂ | Cl | H | p-Cl-phenoxy | H | OMe |
| 1.041 | H | Me | (CH₂)₂ | Me | H | H | H | Me |
| 1.042 | H | Me | (CH₂)₂ | Me | H | Cl | H | Me |
| 1.043 | H | Me | (CH₂)₂ | Me | H | Br | H | Me |
| 1.044 | H | Me | (CH₂)₂ | Me | H | I | H | Me |
| 1.045 | H | Me | (CH₂)₂ | Me | H | Me | H | Me |
| 1.046 | H | Me | (CH₂)₂ | Me | H | C≡C—H | H | Me |
| 1.047 | H | Me | (CH₂)₂ | Me | H | CH═NOMe | H | Me |
| 1.048 | H | Me | (CH₂)₂ | Me | H | OCF₃ | H | Me |
| 1.049 | H | Me | (CH₂)₂ | Me | H | p-Cl-phenoxy | H | Me |
| 1.050 | H | Me | (CH₂)₂ | Br | H | Br | H | Br |
| 1.051 | H | H | (CH₂)₂ | Cl | H | H | H | Cl |
| 1.052 | H | H | (CH₂)₂ | Cl | H | Cl | H | Cl |
| 1.053 | H | H | (CH₂)₂ | Cl | H | Br | H | Cl |
| 1.054 | H | H | (CH₂)₂ | Cl | H | C≡C—H | H | Cl |
| 1.055 | H | H | (CH₂)₂ | Cl | H | CH═NOMe | H | Cl |
| 1.056 | H | H | (CH₂)₂ | Cl | H | OCF₃ | H | Cl |
| 1.057 | H | H | (CH₂)₂ | Cl | H | p-Cl-phenoxy | H | Cl |
| 1.058 | H | H | (CH₂)₂ | Cl | Cl | Cl | H | Cl |
| 1.059 | H | H | (CH₂)₂ | Cl | Cl | Cl | Cl | Cl |
| 1.060 | H | H | (CH₂)₂ | Cl | H | Cl | H | Me |
| 1.061 | H | H | (CH₂)₂ | Cl | H | Cl | H | OMe |
| 1.062 | H | H | (CH₂)₂ | Me | H | Cl | H | Me |
| 1.063 | H | H | (CH₂)₂ | Me | H | Br | H | Me |
| 1.064 | H | H | (CH₂)₂ | Me | H | p-Cl-phenoxy | H | Me |
| 1.065 | H | H | (CH₂)₂ | Br | H | Br | H | Br |
| 1.066 | H | Me | (CH₂)₃ | Cl | H | Cl | H | H |
| 1.067 | H | Me | (CH₂)₃ | Cl | H | H | H | Cl |
| 1.068 | H | Me | (CH₂)₃ | Cl | H | Cl | Cl | H |
| 1.069 | H | Me | (CH₂)₃ | Cl | H | Cl | H | Cl |
| 1.070 | H | Me | (CH₂)₃ | Cl | H | Br | H | Cl |
| 1.071 | H | Me | (CH₂)₃ | Cl | H | I | H | Cl |
| 1.072 | H | Me | (CH₂)₃ | Cl | H | CHF₂ | H | Cl |
| 1.073 | H | Me | (CH₂)₃ | Cl | H | CF₃ | H | Cl |
| 1.074 | H | Me | (CH₂)₃ | Cl | H | C≡C—H | H | Cl |
| 1.075 | H | Me | (CH₂)₃ | Cl | H | C≡C—Me | H | Cl |
| 1.076 | H | Me | (CH₂)₃ | Cl | H | C≡C—t-Bu | H | Cl |
| 1.077 | H | Me | (CH₂)₃ | Cl | H | C≡C-cyclopropyl | H | Cl |
| 1.078 | H | Me | (CH₂)₃ | Cl | H | C≡C-p-Cl-phenyl | H | Cl |
| 1.079 | H | Me | (CH₂)₃ | Cl | H | CH═NOMe | H | Cl |
| 1.080 | H | Me | (CH₂)₃ | Cl | H | CH═NOEt | H | Cl |
| 1.081 | H | Me | (CH₂)₃ | Cl | H | CMe═NOMe | H | Cl |
| 1.082 | H | Me | (CH₂)₃ | Cl | H | OCF₃ | H | Cl |
| 1.083 | H | Me | (CH₂)₃ | Cl | H | OCH₂CH═CHCl₂ | H | Cl |
| 1.084 | H | Me | (CH₂)₃ | Cl | H | p-Cl-phenoxy | H | Cl |
| 1.085 | H | Me | (CH₂)₃ | Cl | H | Cl | Cl | H |
| 1.086 | H | Me | (CH₂)₃ | Cl | Cl | Cl | H | Cl |
| 1.087 | H | Me | (CH₂)₃ | Cl | Cl | Cl | Cl | Cl |
| 1.088 | H | Me | (CH₂)₃ | Cl | H | Cl | H | Me |
| 1.089 | H | Me | (CH₂)₃ | Cl | H | Br | H | Me |
| 1.090 | H | Me | (CH₂)₃ | Cl | H | I | H | Me |
| 1.091 | H | Me | (CH₂)₃ | Cl | H | CF₃ | H | Me |
| 1.092 | H | Me | (CH₂)₃ | Cl | H | C≡C—H | H | Me |
| 1.093 | H | Me | (CH₂)₃ | Cl | H | C≡C—t-Bu | H | Me |
| 1.094 | H | Me | (CH₂)₃ | Cl | H | CH═NOMe | H | Me |
| 1.095 | H | Me | (CH₂)₃ | Cl | H | OCF₃ | H | Me |
| 1.096 | H | Me | (CH₂)₃ | Cl | H | p-Cl-phenoxy | H | Me |
| 1.097 | H | Me | (CH₂)₃ | Cl | H | Cl | H | OMe |
| 1.098 | H | Me | (CH₂)₃ | Cl | H | Br | H | OMe |
| 1.099 | H | Me | (CH₂)₃ | Cl | H | I | H | OMe |
| 1.100 | H | Me | (CH₂)₃ | Cl | H | CF₃ | H | OMe |
| 1.101 | H | Me | (CH₂)₃ | Cl | H | C≡C—H | H | OMe |
| 1.102 | H | Me | (CH₂)₃ | Cl | H | C≡C—t-Bu | H | OMe |
| 1.103 | H | Me | (CH₂)₃ | Cl | H | CH═NOMe | H | OMe |
| 1.104 | H | Me | (CH₂)₃ | Cl | H | OCF₃ | H | OMe |
| 1.105 | H | Me | (CH₂)₃ | Cl | H | p-Cl-phenoxy | H | OMe |
| 1.106 | H | Me | (CH₂)₃ | Me | H | H | H | Me |
| 1.107 | H | Me | (CH₂)₃ | Me | H | Cl | H | Me |
| 1.108 | H | Me | (CH₂)₃ | Me | H | Br | H | Me |
| 1.109 | H | Me | (CH₂)₃ | Me | H | I | H | Me |
| 1.110 | H | Me | (CH₂)₃ | Me | H | Me | H | Me |
| 1.111 | H | Me | (CH₂)₃ | Me | H | C≡C—H | H | Me |
| 1.112 | H | Me | (CH₂)₃ | Me | H | CH═NOMe | H | Me |
| 1.113 | H | Me | (CH₂)₃ | Me | H | OCF₃ | H | Me |
| 1.114 | H | Me | (CH₂)₃ | Me | H | p-Cl-phenoxy | H | Me |
| 1.115 | H | Me | (CH₂)₃ | Br | H | Br | H | Br |
| 1.116 | H | H | (CH₂)₃ | Cl | H | H | H | Cl |
| 1.117 | H | H | (CH₂)₃ | Cl | H | Cl | H | Cl |
| 1.118 | H | H | (CH₂)₃ | Cl | H | Br | H | Cl |
| 1.119 | H | H | (CH₂)₃ | Cl | H | C≡C—H | H | Cl |
| 1.120 | H | H | (CH₂)₃ | Cl | H | CH═NOMe | H | Cl |
| 1.121 | H | H | (CH₂)₃ | Cl | H | OCF₃ | H | Cl |
| 1.122 | H | H | (CH₂)₃ | Cl | H | p-Cl-phenoxy | H | Cl |
| 1.123 | H | H | (CH₂)₃ | Cl | Cl | Cl | H | Cl |
| 1.124 | H | H | (CH₂)₃ | Cl | Cl | Cl | Cl | Cl |
| 1.125 | H | H | (CH₂)₃ | Cl | H | Cl | H | Me |
| 1.126 | H | H | (CH₂)₃ | Cl | H | Cl | H | OMe |
| 1.127 | H | H | (CH₂)₃ | Me | H | Cl | H | Me |
| 1.128 | H | H | (CH₂)₃ | Me | H | Br | H | Me |
| 1.129 | H | H | (CH₂)₃ | Me | H | p-Cl-phenoxy | H | Me |
| 1.130 | H | H | (CH₂)₃ | Br | H | Br | H | Br |
| 1.131 | H | Me | (CH₂)₂O | Cl | H | Cl | H | H |
| 1.132 | H | Me | (CH₂)₂O | Cl | H | H | H | Cl |
| 1.133 | H | Me | (CH₂)₂O | Cl | H | Cl | Cl | H |
| 1.134 | H | Me | (CH₂)₂O | Cl | H | Cl | H | Cl |
| 1.135 | H | Me | (CH₂)₂O | Cl | H | Br | H | Cl |
| 1.136 | H | Me | (CH₂)₂O | Cl | H | I | H | Cl |
| 1.137 | H | Me | (CH₂)₂O | Cl | H | CHF₂ | H | Cl |
| 1.138 | H | Me | (CH₂)₂O | Cl | H | CF₃ | H | Cl |
| 1.139 | H | Me | (CH₂)₂O | Cl | H | C≡C—H | H | Cl |
| 1.140 | H | Me | (CH₂)₂O | Cl | H | C≡C—Me | H | Cl |
| 1.141 | H | Me | (CH₂)₂O | Cl | H | C≡C—t-Bu | H | Cl |
| 1.142 | H | Me | (CH₂)₂O | Cl | H | C≡C-cyclopropyl | H | Cl |
| 1.143 | H | Me | (CH₂)₂O | Cl | H | C≡C-p-Cl-phenyl | H | Cl |
| 1.144 | H | Me | (CH₂)₂O | Cl | H | CH═NOMe | H | Cl |
| 1.145 | H | Me | (CH₂)₂O | Cl | H | CH═NOEt | H | Cl |
| 1.146 | H | Me | (CH₂)₂O | Cl | H | CMe═NOMe | H | Cl |
| 1.147 | H | Me | (CH₂)₂O | Cl | H | OCF₃ | H | Cl |
| 1.148 | H | Me | (CH₂)₂O | Cl | H | OCH₂CH≡CHCl₂ | H | Cl |
| 1.149 | H | Me | (CH₂)₂O | Cl | H | p-Cl-phenoxy | H | Cl |
| 1.150 | H | Me | (CH₂)₂O | Cl | H | Cl | Cl | H |
| 1.151 | H | Me | (CH₂)₂O | Cl | Cl | Cl | H | Cl |
| 1.152 | H | Me | (CH₂)₂O | Cl | Cl | Cl | Cl | Cl |
| 1.153 | H | Me | (CH₂)₂O | Cl | H | Cl | H | Me |
| 1.154 | H | Me | (CH₂)₂O | Cl | H | Br | H | Me |
| 1.155 | H | Me | (CH₂)₂O | Cl | H | I | H | Me |
| 1.156 | H | Me | (CH₂)₂O | Cl | H | CF₃ | H | Me |
| 1.157 | H | Me | (CH₂)₂O | Cl | H | C≡C—H | H | Me |
| 1.158 | H | Me | (CH₂)₂O | Cl | H | C≡C—t-Bu | H | Me |
| 1.159 | H | Me | (CH₂)₂O | Cl | H | CH═NOMe | H | Me |
| 1.160 | H | Me | (CH₂)₂O | Cl | H | OCF₃ | H | Me |
| 1.161 | H | Me | (CH₂)₂O | Cl | H | p-Cl-phenoxy | H | Me |
| 1.162 | H | Me | (CH₂)₂O | Cl | H | Cl | H | OMe |
| 1.163 | H | Me | (CH₂)₂O | Cl | H | Br | H | OMe |
| 1.164 | H | Me | (CH₂)₂O | Cl | H | I | H | OMe |
| 1.165 | H | Me | (CH₂)₂O | Cl | H | CF₃ | H | OMe |
| 1.166 | H | Me | (CH₂)₂O | Cl | H | C≡C—H | H | OMe |
| 1.167 | H | Me | (CH₂)₂O | Cl | H | C≡C—t-Bu | H | OMe |
| 1.168 | H | Me | (CH₂)₂O | Cl | H | CH═NOMe | H | OMe |
| 1.169 | H | Me | (CH₂)₂O | Cl | H | OCF₃ | H | OMe |
| 1.170 | H | Me | (CH₂)₂O | Cl | H | p-Cl-phenoxy | H | OMe |
| 1.171 | H | Me | (CH₂)₂O | Me | H | H | H | Me |
| 1.172 | H | Me | (CH₂)₂O | Me | H | Cl | H | Me |
| 1.173 | H | Me | (CH₂)₂O | Me | H | Br | H | Me |
| 1.174 | H | Me | (CH₂)₂O | Me | H | I | H | Me |
| 1.175 | H | Me | (CH₂)₂O | Me | H | Me | H | Me |
| 1.176 | H | Me | (CH₂)₂O | Me | H | C≡C—H | H | Me |
| 1.177 | H | Me | (CH₂)₂O | Me | H | CH═NOMe | H | Me |
| 1.178 | H | Me | (CH₂)₂O | Me | H | OCF₃ | H | Me |
| 1.179 | H | Me | (CH₂)₂O | Me | H | p-Cl-phenoxy | H | Me |
| 1.180 | H | Me | (CH₂)₂O | Br | H | Br | H | Br |
| 1.181 | H | H | (CH₂)₂O | Cl | H | H | H | Cl |
| 1.182 | H | H | (CH₂)₂O | Cl | H | Cl | H | Cl |
| 1.183 | H | H | (CH₂)₂O | Cl | H | Br | H | Cl |
| 1.184 | H | H | (CH₂)₂O | Cl | H | C≡C—H | H | Cl |
| 1.185 | H | H | (CH₂)₂O | Cl | H | CH═NOMe | H | Cl |
| 1.186 | H | H | (CH₂)₂O | Cl | H | OCF₃ | H | Cl |
| 1.187 | H | H | (CH₂)₂O | Cl | H | p-Cl-phenoxy | H | Cl |
| 1.188 | H | H | (CH₂)₂O | Cl | Cl | Cl | H | Cl |
| 1.189 | H | H | (CH₂)₂O | Cl | Cl | Cl | Cl | Cl |
| 1.190 | H | H | (CH₂)₂O | Cl | H | Cl | H | Me |
| 1.191 | H | H | (CH₂)₂O | Cl | H | Cl | H | OMe |
| 1.192 | H | H | (CH₂)₂O | Me | H | Cl | H | Me |
| 1.193 | H | H | (CH₂)₂O | Me | H | Br | H | Me |
| 1.194 | H | H | (CH₂)₂O | Me | H | p-Cl-phenoxy | H | Me |
| 1.195 | H | H | (CH₂)₂O | Br | H | Br | H | Br |
| 1.196 | H | Me | CH₂OCH₂ | Cl | H | Cl | H | H |
| 1.197 | H | Me | CH₂OCH₂ | Cl | H | H | H | Cl |
| 1.198 | H | Me | CH₂OCH₂ | Cl | H | Cl | Cl | H |
| 1.199 | H | Me | CH₂OCH₂ | Cl | H | Cl | H | Cl |
| 1.200 | H | Me | CH₂OCH₂ | Cl | H | Br | H | Cl |
| 1.201 | H | Me | CH₂OCH₂ | Cl | H | I | H | Cl |
| 1.202 | H | Me | CH₂OCH₂ | Cl | H | CHF₂ | H | Cl |
| 1.203 | H | Me | CH₂OCH₂ | Cl | H | CF₃ | H | Cl |
| 1.204 | H | Me | CH₂OCH₂ | Cl | H | C≡C—H | H | Cl |
| 1.205 | H | Me | CH₂OCH₂ | Cl | H | C≡C—Me | H | Cl |
| 1.206 | H | Me | CH₂OCH₂ | Cl | H | C≡C—t-Bu | H | Cl |
| 1.207 | H | Me | CH₂OCH₂ | Cl | H | C≡C-cyclopropyl | H | Cl |
| 1.208 | H | Me | CH₂OCH₂ | Cl | H | C≡C-p-Cl-phenyl | H | Cl |
| 1.209 | H | Me | CH₂OCH₂ | Cl | H | CH═NOMe | H | Cl |
| 1.210 | H | Me | CH₂OCH₂ | Cl | H | CH═NOEt | H | Cl |
| 1.211 | H | Me | CH₂OCH₂ | Cl | H | CMe═NOMe | H | Cl |
| 1.212 | H | Me | CH₂OCH₂ | Cl | H | OCF₃ | H | Cl |
| 1.213 | H | Me | CH₂OCH₂ | Cl | H | OCH₂CH≡CHCl₂ | H | Cl |
| 1.214 | H | Me | CH₂OCH₂ | Cl | H | p-Cl-phenoxy | H | Cl |
| 1.215 | H | Me | CH₂OCH₂ | Cl | H | Cl | Cl | H |
| 1.216 | H | Me | CH₂OCH₂ | Cl | Cl | Cl | H | Cl |
| 1.217 | H | Me | CH₂OCH₂ | Cl | Cl | Cl | Cl | Cl |
| 1.218 | H | Me | CH₂OCH₂ | Cl | H | Cl | H | Me |
| 1.219 | H | Me | CH₂OCH₂ | Cl | H | Br | H | Me |
| 1.220 | H | Me | CH₂OCH₂ | Cl | H | I | H | Me |
| 1.221 | H | Me | CH₂OCH₂ | Cl | H | CF₃ | H | Me |
| 1.222 | H | Me | CH₂OCH₂ | Cl | H | C≡C—H | H | Me |
| 1.223 | H | Me | CH₂OCH₂ | Cl | H | C≡C—t-Bu | H | Me |
| 1.224 | H | Me | CH₂OCH₂ | Cl | H | CH═NOMe | H | Me |
| 1.225 | H | Me | CH₂OCH₂ | Cl | H | OCF₃ | H | Me |
| 1.226 | H | Me | CH₂OCH₂ | Cl | H | p-Cl-phenoxy | H | Me |
| 1.227 | H | Me | CH₂OCH₂ | Cl | H | Cl | H | OMe |
| 1.228 | H | Me | CH₂OCH₂ | Cl | H | Br | H | OMe |
| 1.229 | H | Me | CH₂OCH₂ | Cl | H | I | H | OMe |
| 1.230 | H | Me | CH₂OCH₂ | Cl | H | CF₃ | H | OMe |
| 1.231 | H | Me | CH₂OCH₂ | Cl | H | C≡C—H | H | OMe |
| 1.232 | H | Me | CH₂OCH₂ | Cl | H | C≡C—t-Bu | H | OMe |
| 1.233 | H | Me | CH₂OCH₂ | Cl | H | CH═NOMe | H | OMe |
| 1.234 | H | Me | CH₂OCH₂ | Cl | H | OCF₃ | H | OMe |
| 1.235 | H | Me | CH₂OCH₂ | Cl | H | p-Cl-phenoxy | H | OMe |
| 1.236 | H | Me | CH₂OCH₂ | Me | H | H | H | Me |
| 1.237 | H | Me | CH₂OCH₂ | Me | H | Cl | H | Me |
| 1.238 | H | Me | CH₂OCH₂ | Me | H | Br | H | Me |
| 1.239 | H | Me | CH₂OCH₂ | Me | H | I | H | Me |
| 1.240 | H | Me | CH₂OCH₂ | Me | H | Me | H | Me |
| 1.241 | H | Me | CH₂OCH₂ | Me | H | C≡C—H | H | Me |
| 1.242 | H | Me | CH₂OCH₂ | Me | H | CH═NOMe | H | Me |
| 1.243 | H | Me | CH₂OCH₂ | Me | H | OCF₃ | H | Me |
| 1.244 | H | Me | CH₂OCH₂ | Me | H | p-Cl-phenoxy | H | Me |
| 1.245 | H | Me | CH₂OCH₂ | Br | H | Br | H | Br |
| 1.246 | H | H | CH₂OCH₂ | Cl | H | H | H | Cl |
| 1.247 | H | H | CH₂OCH₂ | Cl | H | Cl | H | Cl |
| 1.248 | H | H | CH₂OCH₂ | Cl | H | Br | H | Cl |
| 1.249 | H | H | CH₂OCH₂ | Cl | H | C≡C—H | H | Cl |
| 1.250 | H | H | CH₂OCH₂ | Cl | H | CH═NOMe | H | Cl |
| 1.251 | H | H | CH₂OCH₂ | Cl | H | OCF₃ | H | Cl |
| 1.252 | H | H | CH₂OCH₂ | Cl | H | p-Cl-phenoxy | H | Cl |
| 1.253 | H | H | CH₂OCH₂ | Cl | Cl | Cl | H | Cl |
| 1.254 | H | H | CH₂OCH₂ | Cl | Cl | Cl | Cl | Cl |
| 1.255 | H | H | CH₂OCH₂ | Cl | H | Cl | H | Me |
| 1.256 | H | H | CH₂OCH₂ | Cl | H | Cl | H | OMe |
| 1.247 | H | H | CH₂OCH₂ | Me | H | Cl | H | Me |
| 1.258 | H | H | CH₂OCH₂ | Me | H | Br | H | Me |
| 1.259 | H | H | CH₂OCH₂ | Me | H | p-Cl-phenoxy | H | Me |
| 1.260 | H | H | CH₂OCH₂ | Br | H | Br | H | Br |

In formula Ia, R₃ represents hydrogen or cyclopropyl. In the compounds 1.001-1.260 of Table 1, R₃ represents hydrogen.
“Me” means the methyl group.

The invention is further illustrated by the preferred individual compounds of formula (V)

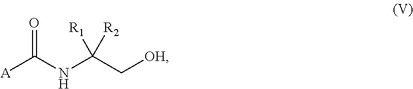

which are listed below in Table 2.

TABLE 2
Preferred compounds of formula V:

| Cpd No. | R₁ | R₂ | A |
|---|---|---|---|
| Z1.1 | H | H | 3-difluoromethyl-1-methyl-1H-pyrazol-4-yl |
| Z1.2 | Me | H | 3-difluoromethyl-1-methyl-1H-pyrazol-4-yl |
| Z1.3 | H | H | 3-trifluoromethyl-1-methyl-1H-pyrazol-4-yl |
| Z1.4 | Me | H | 3-trifluoromethyl-1-methyl-1H-pyrazol-4-yl |

### Table 3: Characterising Data:

Table 3 shows selected melting point and selected NMR data for compounds of Table 1. CDCl₃ is used as the solvent for NMR measurements, unless otherwise stated. If a mixture of solvents is present, this is indicated as, for example: CDCl₃/d₆-DMSO). No attempt is made to list all characterising data in all cases.

In Table 3 and throughout the description that follows, temperatures are given in degrees Celsius; “NMR” means nuclear magnetic resonance spectrum; MS stands for mass spectrum; “%” is percent by weight, unless corresponding concentrations are indicated in other units. The following abbreviations are used throughout this description:

| | |
|---|---|
| m.p. = melting point | b.p. = boiling point. |
| S = singlet | br = broad |
| d = doublet | dd = doublet of doublets |
| t = triplet | q = quartet |
| m = multiplet | ppm = parts per million |

TABLE 3

| Cpd | 1H-NMR data: ppm | | | |
|---|---|---|---|---|
| No. | (multiplicity/number of Hs) | MS [M + H]⁺ | m.p. (° C.) | LCMS data |
| 1.004 | 7.93 (s, 1H); 7.32 (s, 1H); | 410/412/414 | 100 | |
| | 6.94-6.67 (t, J = 54, 1H); 6.26 (s, 1H); | | | |
| | 4.31-4.24 (m, 1H); 3.92 (s, 3H); 2.99-2.92 (m, | | | |
| | 2H); 1.79-1.67 (m, 2H); 1.29-1.27 (d, | | | |
| | J = 6.4, 3H) | | | |
| 1.069 | 7.89 (s, 1H); 7.27 (s, 2H); 6.92-6.65 (m, 1H); | 424/426/428 | 120 | |
| | 6.14 (s, 1H); 4.22-4.14 (m, 1H); 3.91 (s, 3H); | | | |
| | 2.90-2.86 (m, 2H); 1.62-1.60 (m, 4H); | | | |
| | 1.20 (d, J = 6.8 Hz, 3H). | | | |
| 1.197 | 1.37-1.39 (d, 3H, CH₃), 3.52-3.58 (m, 2H, CH₂), | 392/394/396 | oil | |
| | 3.91 (s, 3H, CH₃), 4.32-4.41 (m, 1H, CH), | | | |
| | 4.59 (s, 2H, CH₂), 6.57 (s, 1H, NH), | | | |
| | 6.71-6.97 (t, 1H, CHF2), 7.23 (d × d, 1H, Ar—H), | | | |
| | 7.36 (d, 1H, Ar—H), 7.42 (d, 1H, Ar—H), | | | |
| | 7.88 (t, 1H, Py—H). | | | |
| 1.001 | 1.25 (d, 3H), 1.72-1.83 (m, 2H), | | 108 | 375.95/377.83/ |
| | 2.69-2.84 (m, 2H), 3.92 (s, 3H), 4.16-4.23 (m, 1H), | | | 378.57 |
| | 6.21 (s, 1H), 6.67-6.94 (t, 3H), 7.15 (d, 2H), | | | |
| | 7.33 (d, 1H), 7.92 (s, 1H) | | | |
| 1.052 | 1.81-1.88 (m, 2H), 2.94-2.98 (t, 2H), | | 91-93 | 395.97/397.75 |
| | 3.47-3.52 (q, 2H), 3.91 (s, 3H), 6.46 (s, 1H), | | | |
| | 6.67-6.94 (t, 1H), 7.29 (s, 2H), 7.92 (s, 1H) | | | |
| 1.134 | 1.33-1.35 (d, 3H), 2.00-2.07 (m, 1H), | | 78-80 | 425.95/427.31/ |
| | 2.12-2.17 (m, 1H), 3.89 (s, 3H), 4.02-4.07 (m, 1H), | | | 428.4 |
| | 4.12-4.18 (m, 1H), 4.40-4.44 (m, 1H), | | | |
| | 6.46 (s, 1H), 6.70-6.97 (t, 1H), 7.28 (s, 2H), | | | |
| | 7.85 (s, 1H) | | | |
| 1.171 | 1.18 (d, 3H), 1.91 (m, 2H), 2.2 (s, 6H), | | resin | 352.01 |
| | 3.69 (m, 2H), 3.90 (s, 3H), 4.19 (m, 1H), | | | |
| | 6.97 (d, 2H), 7.18 (t, 1H), 8.00 (d, 1H), | | | |
| | 8.26 (s, 1H) | | | |
| 1.182 | 2.09-2.16 (m, 2H), 3.70-3.74 (q, 2H), | | 98-100 | 411.95/413.72/ |
| | 3.91 (s, 3H), 4.07-4.10 (t, 2H), 6.69 (s, 1H), | | | 415.64 |
| | 6.69-6.96 (t, 1H), 7.31 (s, 2H), 7.88 (s, 1H) | | | |

### Formulation Examples for Compounds of Formula I

### Example F-1.1 to F-1.2

### Emulsifiable Concentrates

| Components | F-1.1 | F-1.2 |
|---|---|---|
| compound of Table 1 | 25% | 50% |
| calcium dodecylbenzenesulfonate | 5% | 6% |
| castor oil polyethylene glycol ether | 5% | — |
| (36 mol ethylenoxy units) | | |
| tributylphenolpolyethylene glycol ether | — | 4% |
| (30 mol ethylenoxy units) | | |
| cyclohexanone | — | 20% |
| xylene mixture | 65% | 20% |

Emulsions of any desired concentration can be prepared by diluting such concentrates with water.

### Example F-2

### Emulsifiable Concentrate

| Components | F-2 |
|---|---|
| compound of Table 1 | 10% |
| octylphenolpolyethylene glycol ether | 3% |
| (4 to 5 mol ethylenoxy units) | |
| calcium dodecylbenzenesulfonate | 3% |
| castor oil polyglycol ether | 4% |
| (36 mol ethylenoxy units) | |
| cyclohexanone | 30% |
| xylene mixture | 50% |

Emulsions of any desired concentration can be prepared by diluting such concentrates with water.

### Examples F-3.1 to F-3.4

### Solutions

| Components | F-3.1 | F-3.2 | F-3.3 | F-3.4 |
|---|---|---|---|---|
| compound of Table 1 | 80% | 10% | 5% | 95% |
| propylene glycol monomethyl ether | 20% | — | — | — |
| polyethylene glycol (relative molecular | — | 70% | — | — |
| mass: 400 atomic mass units) | | | | |
| N-methylpyrrolid-2-one | — | 20% | — | — |
| epoxidised coconut oil | — | — | 1% | 5% |
| benzin (boiling range: 160-190°) | — | — | 94% | — |

The solutions are suitable for use in the form of microdrops.

### Examples F-4.1 to F-4.4

### Granulates

| Components | F-4.1 | F-4.2 | F-4.3 | F-4.4 |
|---|---|---|---|---|
| compound of Table 1 | 5% | 10% | 8% | 21% |
| kaolin | 94% | — | 79% | 54% |
| highly dispersed silicic acid | 1% | — | 13% | 7% |
| attapulgite | — | 90% | — | 18% |

The novel compound is dissolved in dichloromethane, the solution is sprayed onto the carrier and the solvent is then removed by distillation under vacuum.

### Examples F-5.1 and F-5.2

### Dusts

| Components | F-5.1 | F-5.2 |
|---|---|---|
| compound of Table 1 | 2% | 5% |
| highly dispersed silicic acid | 1% | 5% |
| talcum | 97% | — |
| kaolin | — | 90% |

Ready for use dusts are obtained by intimately mixing all components.

### Examples F-6.1 to F-6.3

### Wettable Powders

| Components | F-6.1 | F-6.2 | F-6.3 |
|---|---|---|---|
| compound of Table 1 | 25% | 50% | 75% |
| sodium lignin sulfonate | 5% | 5% | — |
| sodium lauryl sulfate | 3% | — | 5% |
| sodium diisobutylnaphthalene sulfonate | — | 6% | 10% |
| octylphenolpolyethylene glycol ether | — | 2% | — |
| (7 to 8 mol ethylenoxy units) | | | |
| highly dispersed silicic acid | 5% | 10% | 10% |
| kaolin | 62% | 27% | — |

All components are mixed and the mixture is thoroughly ground in a suitable mill to give wettable powders which can be diluted with water to suspensions of any desired concentration.

### Example F7

### Flowable Concentrate for Seed Treatment

| | |
|---|---|
| compound of Table 1 | 40% |
| propylene glycol | 5% |
| copolymer butanol PO/EO | 2% |
| tristyrenephenole with 10-20 moles EO | 2% |
| 1,2-benzisothiazolin-3-one (in the form of a | 0.5% |
| 20% solution in water) | |
| monoazo-pigment calcium salt | 5% |
| Silicone oil (in the form of a 75% | 0.2% |
| emulsion in water) | |
| Water | 45.3% |

The finely ground active ingredient is intimately mixed with the adjuvants, giving a suspension concentrate from which suspensions of any desired dilution can be obtained by dilution with water. Using such dilutions, living plants as well as plant propagation material can be treated and protected against infestation by microorganisms, by spraying, pouring or immersion.

### Biological Examples

### Fungicidal Action

### Example B-1

### Action Against Erysiphe graminis f.sp. Tritici (Wheat Powdery Mildew)

Wheat leaf segments are placed on agar in multiwell plates (24-well format) and sprayed with test solutions (0.02% active ingredient). After drying, the leaf disks are inoculated with a spore suspension of the fungus. After appropriate incubation the activity of a compound is assessed 7 days after inoculation as preventive fungicidal activity. Compounds 1.001, 1.004, 1.052, 1.069, 1.134, and 1.182 show good activity in this test (at least 50% inhibition).

### Example B-2

### Action Against Pyrenophora teres (Net Blotch) on Barley

Barley leaf segments are placed on agar in multiwell plates (24-well format) and sprayed with test solutions (0.02% active ingredient). After drying, the leaf disks are inoculated with a spore suspension of the fungus. After appropriate incubation the activity of a compound is assessed 4 days after inoculation as preventive fungicidal activity. Compounds 1.001, 1.004, 1.052, 1.069, 1.134, 1.171, 1.182 and 1.197 show good activity in this test (at least 50% inhibition).

### Example B-3

### Action Against Botrytis cinerea—Fungal Growth Assay

Conidia of the fungus from cryogenic storage are directly mixed into nutrient broth (PDB potato dextrose broth). After placing a (DMSO) solution of the test compounds (0.02% active ingredient) into a microtiter plate (96-well format) the nutrient broth containing the fungal spores is added. The test plates are incubated at 24° C. and the inhibition of growth is measured photometrically after 3-4 days. The activity of a compound is expressed as fungal growth inhibition. Compounds 1.001, 1.004, 1.052, 1.134, 1.182 and 1.197 show good activity in this test (at least 50% inhibition).

### Example B-4

### Action Against Mycosphaerella arachidis (Early Leaf Spot of Groundnut Cercospora Arachidicola [Anamorph])—Fungal Growth Assay

Conidia of the fungus from cryogenic storage are directly mixed into nutrient broth (PDB potato dextrose broth). After placing a (DMSO) solution of the test compounds (0.02% active ingredient) into a microtiter plate (96-well format) the nutrient broth containing the fungal spores is added. The test plates are incubated at 24° C. and the inhibition of growth is measured photometrically after 6-7 days. The activity of a compound is expressed as fungal growth inhibition. Compounds 1.001, 1.004, 1.052, 1.069, 1.134, 1.171, 1.182 and 1.197 show good activity in this test (at least 50% inhibition).

### Example B-5

### Action Against Septoria tritici—Fungal Growth Assay

Conidia of the fungus from cryogenic storage are directly mixed into nutrient broth (PDB potato dextrose broth). After placing a (DMSO) solution of the test compounds (0.02% active ingredient) into a microtiter plate (96-well format) the nutrient broth containing the fungal spores is added. The test plates are incubated at 24° C. and the inhibition of growth is determined photometrically after 72 hrs. The activity of a compound is expressed as fungal growth inhibition. Compounds 1.001, 1.004, 1.052, 1.069, 1.134, 1.171, 1.182 and 1.197 show good activity in this test (at least 50% inhibition).

### Example B-6

### Action Against Monographella nivalis (Anamorph: Fusarium nivale, Microdochium nivale; Snow Mould)—Fungal Growth Assay

Conidia of the fungus from cryogenic storage are directly mixed into nutrient broth (PDB potato dextrose broth). After placing a DMSO-solution of the test compounds (0.02% active ingredient) into a microtiter plate (96-well format) the nutrient broth containing the fungal spores is added. The test plates are incubated at 24° C. and the inhibition of growth is measured photometrically after 72 hrs. Compounds 1.001, 1.004, 1.052, 1.069, 1.134, 1.171, 1.182 and 1.197 show good activity in this test (at least 50% inhibition).

### Example B-7

### Curative Action Against Puccinia recondita (Brown Rust) on Wheat

Wheat leaf segments are placed on agar in multiwell plates (24-well format) and inoculated with a spore suspension of the fungus. One day after inoculation the leaf segments are sprayed with test solutions (0.02% active ingredient). After appropriate incubation the activity of a compound is assessed 8 days after inoculation as curative fungicidal activity. Compounds 1.004 and 1.052 show good activity in this test (at least 50% inhibition).

### Example B-8

### Action Against Leptosphaeria nodorum (Septoria nodorum; Glume Blotch) on Wheat

Wheat leaf segments are placed on agar in multiwell plates (24-well format) and sprayed with test solutions (0.02% active ingredient). After drying, the leaf disks are inoculated with a spore suspension of the fungus. After appropriate incubation the activity of a compound is assessed 4 days after inoculation as preventive fungicidal activity. Compounds 1.001, 1.004, 1.069, 1.134, and 1.182 show good activity in this test (at least 50% inhibition).

What is claimed is:

1. A compound of formula I 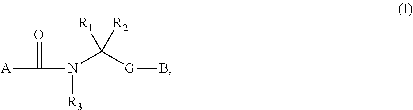
wherein
R₁ is hydrogen or C₁-C₄alkyl; R₂ is hydrogen or C₁-C₄alkyl;
R₃ is hydrogen or C₃-C₇cycloalkyl;
B is the group B₁ 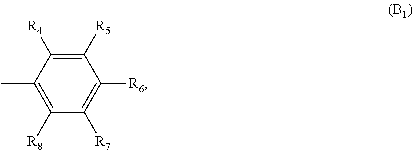
wherein
R₄ is hydrogen, halogen or C₁-C₆alkyl;
R₅ is hydrogen or halogen;
R₆ is hydrogen, halogen, C₁-C₆alkyl, C₂-C₆alkenyl, C₃-C₆alkinyl, C₃-C₆cycloalkyl-C₃-C₆alkinyl, halophenoxy, halophenyl-C₃-C₆alkinyl, C(C₁-C₄alkyl)═NO—C₁-C₄alkyl, C₁-C₆haloalkyl, C₁-C₆haloalkoxy, C₂-C₆haloalkenyl, or C₂-C₆haloalkenyloxy;
R₇ is hydrogen or halogen;
R₈ is hydrogen, halogen, C₁-C₆alkyl, C₁-C₆alkoxy,
G is selected from the group consisting of (CH₂)₂, (CH₂)₃, (CH₂)₂O and CH₂OCH₂;
A is the group A₁ 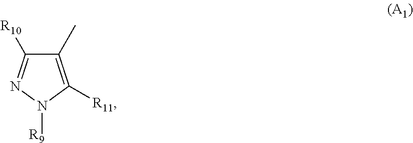
wherein
R₉ is hydrogen or C₁-C₄alkyl;
R₁₀ is C₁-C₄alkyl, C₁-C₄haloalkyl or halogen;
R₁₁ is hydrogen or halogen; and agronomically acceptable salts/isomers/structural isomers/stereoisomers/diastereoisomers/enantio-mers/tautomers and N-oxides of those compounds.

2. A compound according to claim 1, wherein R₁₀ is difluoromethyl or trifluoromethyl.

3. A compound according to claim 1, wherein G is selected from (CH₂)₂, (CH₂)₃ and CH₂OCH₂.

4. A compound according to claim 1, wherein R₁ is hydrogen.

5. A compound according to claim 1, wherein R₂ is hydrogen or methyl.

6. A compound according to claim 1, wherein R₉ is methyl; R₁₀ is C₁-C₄haloalkyl and R₁₁ is hydrogen.

7. A compound of formula Va 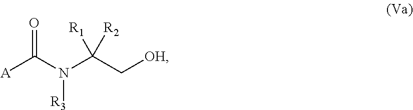
wherein A, R₁, R₂ and R₃ are as defined for formula I in claim 1.

8. A compound of formula VIIIa 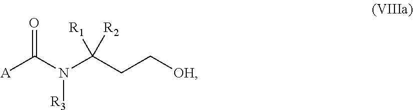
wherein A, R₁, R₂ and R₃ are as defined for formula I in claim 1.

9. A compound of formula XB 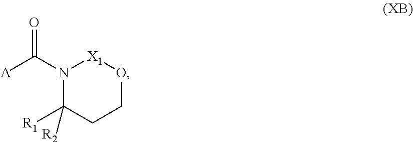
wherein A, R₁ and R₂ are as defined for formula I in claim 1 and X₁ is —S(O)— or —S(O)₂—.

10. A compound of formula IIb2 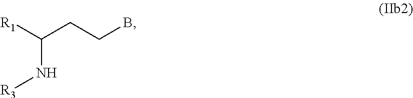
wherein B, R₁ and R₃ are as defined for formula I in claim 1.

11. A compound of formula IIb3 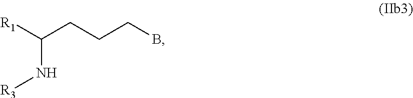
wherein B, R₁ and R₃ are as defined for formula I in claim 1.

12. A method of controlling or preventing infestation of useful plants by phytopathogenic microorganisms, wherein a compound of formula I according to claim 1 or a composition, comprising this compound as active ingredient, is applied to the plants, to parts thereof or the locus thereof.

13. A composition for controlling and protecting against phytopathogenic microorganisms, comprising a compound of formula I according to claim 1 and an inert carrier.
